(19) United States
(12) Patent Application Publication
(10) Pub. No.: US 2024/0366091 A1
(43) Pub. Date: Nov. 7, 2024

(54) ELECTRONIC DEVICE FOR BODY TEMPERATURE MEASUREMENT AND OPERATION METHOD THEREOF

(71) Applicant: Samsung Electronics Co., Ltd., Suwon-si (KR)

(72) Inventors: Hongji LEE, Suwon-si (KR); Jeongmin PARK, Suwon-si (KR)

(21) Appl. No.: 18/774,316

(22) Filed: Jul. 16, 2024

Related U.S. Application Data
(63) Continuation of application No. PCT/KR2022/019235, filed on Nov. 30, 2022.

(30) Foreign Application Priority Data
Jan. 24, 2022 (KR) 10-2022-0010056
Mar. 23, 2022 (KR) 10-2022-0035753

(51) Int. Cl. A61B 5/01; A61B 5/00; G01K 13/20

(52) U.S. Cl. CPC A61B 5/01; A61B 5/681; A61B 5/6844; A61B 5/7282; A61B 5/7475; G01K 13/20; A61B 2560/0252

(57) ABSTRACT

An electronic device for body temperature measurement and an operation method thereof are provided. The electronic device includes memory storing one or more computer programs, a communication circuit, at least one sensor, and one or more processors communicatively coupled to the memory, the communication circuit, and the at least one sensor, wherein the one or more computer programs include computer-executable instructions that, when executed by the one or more processors individually or collectively, cause the electronic device to detect, through the communication circuit or the at least one sensor, a plurality of electronic devices which a user is wearing or with which a user is coming in contact, determine a representative electronic device from among the plurality of electronic devices on the basis of measurement context information, and acquire body temperature information of the user by using the representative electronic device.

### CROSS-REFERENCE TO RELATED APPLICATION(S)

This application is a continuation application, claiming priority under § 365 (c), of an International application No. PCT/KR2022/019235, filed on Nov. 30, 2022, which is based on and claims the benefit of a Korean patent application number 10-2022-0010056, filed on Jan. 24, 2022, in the Korean Intellectual Property Office, and of a Korean patent application number 10-2022-0035753, filed on Mar. 23, 2022, in the Korean Intellectual Property Office, the disclosure of each of which is incorporated by reference herein in its entirety.

### BACKGROUND

### 1. Field

The disclosure relates to an electronic device for body temperature measurement and an operation method thereof.

### 2. Description of Related Art

As the interest in health and medical care is increasing, biometric data measurement functions are commonplace even in personal electronic devices (for example, smartphones, smart watches). An electronic device may use a biometric sensor to measure biometric data. For example, a biometric sensor may be disposed in a portion of an electronic device that is in contact with or is close to user's body, and biometric data, such as body temperature, blood pressure, blood glucose, blood volume, heart rate, electrocardiogram, may be measured by using such a biometric sensor.

A personal electronic device may be daily used by a user, and may have a mechanical characteristic in the capability of carrying or wearing. Due to such a mechanical characteristic, it is possible for a person electronic device to monitor biometric data of a user easily and naturally in user's daily life.

The above information is presented as background information only to assist with an understanding of the disclosure. No determination has been made, and no assertion is made, as to whether any of the above might be applicable as prior art with regard to the disclosure.

### SUMMARY

Body temperature, which is biometric data, is one of vital signs important to human bodies. Body temperature may change to some extent depending on not only biological factors, such as human body activity level, age, or sex of a target body, but also environmental factors such as measurement time or temperature, but may be maintained within a predetermined range by human body's thermoregulation. An abnormal change in body temperature is highly related to health problems or diseases, and thus may be utilized as a key indicator for health management or prognosis of various diseases.

An electronic device may convert a skin temperature detected through a sensor embedded therein into a body temperature by using an algorithm. Skin temperature may be influenced by an environmental factor, and accordingly, accuracy of body temperature measurement may be degraded.

Recently, it is common for individuals to possess multiple electronic devices, and accordingly, it is possible to measure body temperature using multiple electronic devices. However, the efficient body temperature measurement methods using multiple electronic devices are insufficient.

Aspect of the disclosure are to address at least the above-mentioned problems and/or disadvantages and to provide at least the advantages described below. Accordingly, an aspect of the disclosure is to provide an electronic device which is capable of measuring body temperature efficiently by using multiple electronic devices that are easily accessible by an individual in a daily life, and an operation method thereof.

Another aspect of the disclosure is to provide an electronic device which is capable of providing highly accurate body temperature information to a user by using multiple electronic devices, and an operation method thereof.

Another aspect of the disclosure is to provide an electronic device which is capable of continuously providing highly accurate body temperature information to a user by adaptively responding to various measurement environments or a real-time change in a measurement environment, and an operation method thereof.

Additional aspects will be set forth in part in the description which follows and, in part, will be apparent from the description, or may be learned by practice of the presented embodiments.

In accordance with an aspect of the disclosure, an electronic device is provided. The electronic device includes memory storing one or more computer programs, a communication circuit, at least one sensor, and one or more processors communicatively coupled to the memory, the communication circuit, and the at least one sensor, wherein the one or more computer programs include computer-executable instructions that, when executed by the one or more processors individually or collectively, cause the electronic device to detect a plurality of electronic devices with which a user is in contact or which a user is wearing through the communication circuit or the at least one sensor, determine a representative electronic device among the plurality of electronic devices, based on measurement context information, and acquire body temperature information of the user by using the representative electronic device.

In accordance with another aspect of the disclosure, an operation method of an electronic device is provided. The operation method includes detecting a plurality of electronic devices with which a user is in contact or which a user is wearing, determining a representative electronic device among the plurality of electronic devices, based on measurement context information, and acquiring body temperature information of the user by using the representative electronic device.

In accordance with another aspect of the disclosure, one or more non-transitory computer-readable storage media storing one or more computer programs including computer-executable instructions that, when executed by one or more processors of an electronic device individually or collectively, cause the electronic device to perform operations are provided. The operations include detecting a plurality of electronic devices with which a user is in contact or which a user is wearing, determining a representative electronic device among the plurality of electronic devices, based on measurement context information, and acquiring body temperature information of the user by using the representative electronic device.

According to various embodiments, efficient body temperature measurement is possible by using multiple electronic devices that are easily accessible by an individual in a daily life.

According to various embodiments, highly accurate body temperature information is provided to a user by using multiple electronic devices.

According to various embodiments, highly accurate body temperature information is continuously provided to a user by adaptively responding to various measurement environments or real-time change in a measurement environment.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2 is a block diagram of an electronic device according to an embodiment of the disclosure;

FIG. 3 is a view illustrating use states of multiple electronic devices according to an embodiment of the disclosure;

FIG. 4 is a view illustrating body temperature differences by body positions according to external temperatures to explain an operation method of an electronic device according to an embodiment of the disclosure;

FIGS. 5A, 5B, and 5C are views illustrating sensor types and sensor arrangement structures applicable to an electronic device according to various embodiments of the disclosure;

FIG. 6 is a flowchart illustrating an operation method of an electronic device according to an embodiment of the disclosure;

FIG. 7 is another flowchart illustrating an operation method of an electronic device according to an embodiment of the disclosure;

FIG. 8 is a graph illustrating body temperature information provided by an electronic device according to an embodiment of the disclosure; and

FIGS. 9A and 9B are views illustrating examples of user interfaces related to a body temperature measurement function of an electronic device according to various embodiments of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

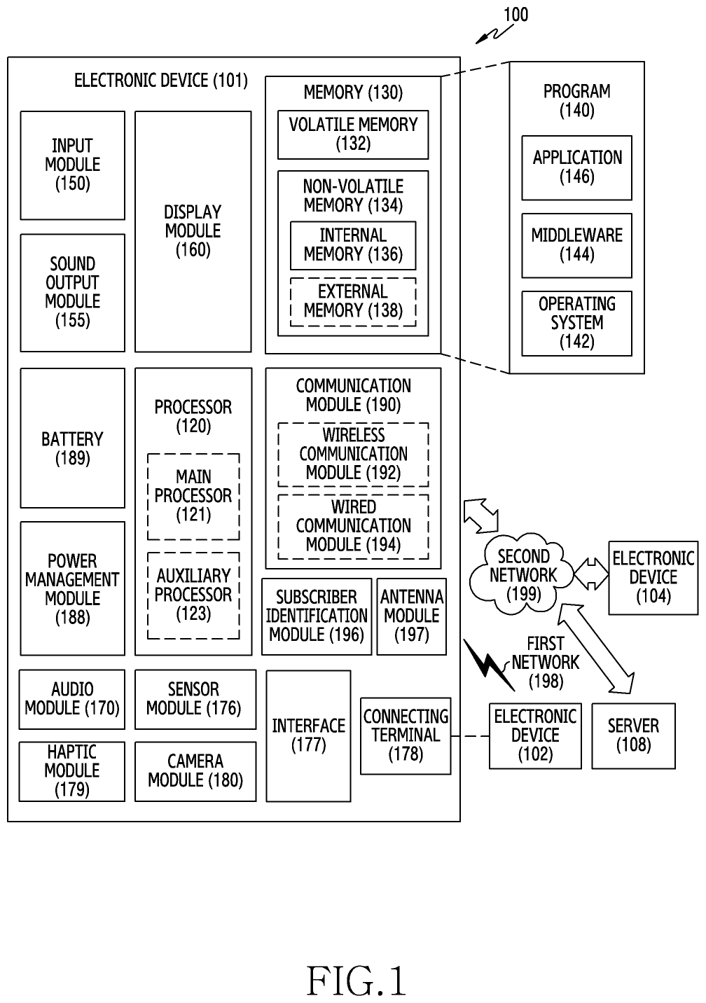

FIG. 1 is a block diagram illustrating an electronic device 101 in a network environment 100 according to an embodiment of the disclosure.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

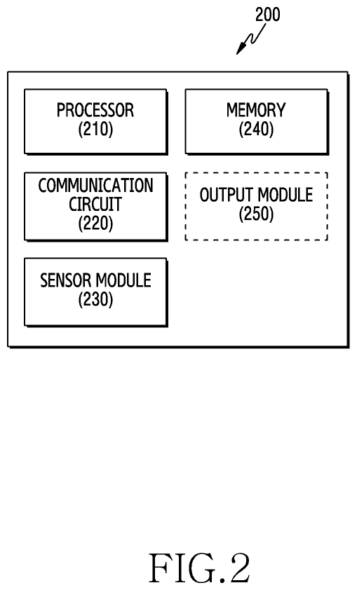

FIG. 2 is a block diagram of an electronic device according to an embodiment of the disclosure.

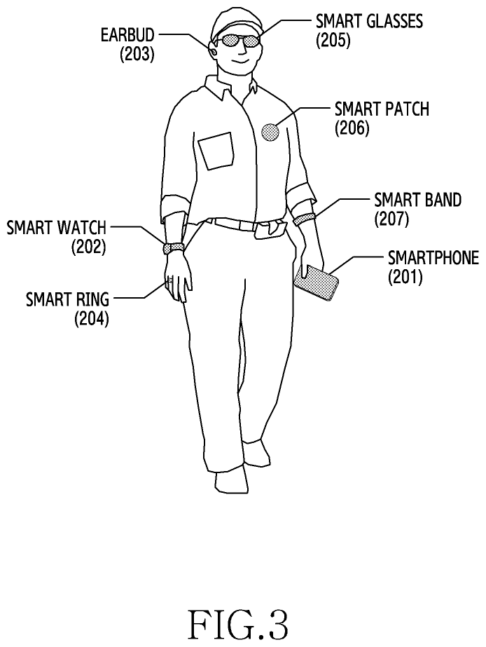

According to an embodiment, the electronic device 200 may be implemented in various types. For example, the electronic device 200 may be implemented in a portable and contactable (or grippable) type (for example, a smartphone 201 of FIG. 3). In another example, the electronic device 200 may be implemented by a wearable electronic device of a wearable (or attachable) type (for example, a smart watch 202, an earbud 203, a smart ring 204, smart glasses 205, a smart patch 206, or a smart band 207 of FIG. 3).

Referring to FIG. 2, the electronic device 200 according to an embodiment may include a processor 210, a communication circuit 220, a sensor module 230, and memory 240. The electronic device 200 may further include an output module 250. The electronic device may omit at least one of the components or may further include other components.

The processor 210, the communication circuit 220, the sensor module 230, the memory 240 and/or the output module 250 included in the electronic device 200 may be electrically and/or operably connected with one another to exchange signals (for example, commands or data).

In an embodiment, the electronic device 200 may include at least part of the electronic device 101 illustrated in FIG. 1. For example, the processor 210 may correspond to the processor 120 (one of 120, 121, or 123) of FIG. 1. The communication circuit 220 may correspond to the communication module 190 of FIG. 1. The sensor module 230 may correspond to the sensor module 176 of FIG. 1 or may include a part thereof. The memory 240 may include at least part of the memory 130 of FIG. 1. The output module 250 may include at least part of the display module 160, the audio module 170, the sound output module 155, and the haptic module 179 of FIG. 1.

In an embodiment, the processor 210 may execute and/or control various functions supported in the electronic device 200. The processor 210 may control at least part of the communication circuit 220, the sensor module 230, the memory 240, and the output module 250. The processor 210 may execute an application by executing a code written by a programming language stored in the memory 240, and may control a variety of hardware of the electronic device 200.

For example, the processor 210 may execute an application (hereinafter, may be referred to as “App”) (for example, a body temperature application, a health care application, a fitness application, a sleep application), and may provide a body temperature monitoring function and/or a body temperature measurement function by using the application. The application executed in the electronic device 200 (for example, the smartphone 201 of FIG. 3) may independently operate or may operate by interlocking with an external electronic device (for example, the smart watch 202 of FIG. 3, the electronic device 102, 104 of FIG. 1 or the server 108 of FIG. 1).

In an embodiment, the processor 210 may include at least one processor. For example, the processor 210 may include a main processor which is physically separated and performs high-performance processing, and a sub processor which performs low-power performance. In another example, the processor 210 may include at least one of an application processor and a sensor hub processor.

The processor 210 may perform a body temperature measurement function and/or a body temperature monitoring function. For example, the processor 210 (for example, a sub processor, a sensor hub processor) may be continuously or periodically connected with a body temperature sensor to perform the body temperature monitoring function. In another example, the processor 210 may process signals detected by the body temperature sensor while switching between the high-performance main processor and the low-power sub processor according to a context.

In an embodiment, the processor 210 may acquire body temperature information by processing signals detected by at least one sensor (for example, the body temperature sensor) in the sensor module 230. The processor 210 may provide (or output) a user interface related to the body temperature information through the output module 250. For example, the processor 210 may display a screen related to the body temperature information through a display of the output module 250, or may output a sound or vibration feedback related to the body temperature information through an audio circuit or a haptic circuit of the output module 250.

In an embodiment, instructions stored in the memory 240 may be executed to cause the processor 210 to perform operations.

In an embodiment, the communication circuit 220 may include a wireless communication module (for example, the wireless communication module 192 of FIG. 1). For example, the wireless communication module may include at least one of a cellular communication module, a short-range wireless communication module, or a global navigation satellite system (GNSS) communication module.

In an embodiment, the communication circuit 220 may support short-range wireless communication connection of the electronic device 200. For example, the communication circuit 220 may support short-range wireless communication network (for example, the first network 198 of FIG. 1) connection between the electronic device 200 (for example, the smartphone 201 of FIG. 3) and an external electronic device (for example, the smart watch 202 of FIG. 3 or the electronic device 102, 104 of FIG. 1). For example, the communication circuit 220 may support short-range wireless communication network (for example, Bluetooth, Bluetooth low energy (BLE), wireless fidelity (WiFi), near field communication (NFC), infrared data association (IrDA), or ultra-wideband (UWB)) connection between the electronic device 200 and an external electronic device, and may transmit a body temperature measurement result and/or a body temperature monitoring result of the electronic device 200 (for example, the smartphone 201 of FIG. 3) to the external electronic device (for example, the smart watch 202 of FIG. 3 or the electronic device 102, 104 of FIG. 1).

In an embodiment, the communication circuit 220 may support long-range wireless communication network (for example, the second network 199 of FIG. 1) connection of the electronic device 200 (for example, the smartphone 201 or the smart watch 202 of FIG. 3). For example, the communication circuit 220 may communicate with a server (for example, the server 108 of FIG. 1) which supports execution of an application for the body temperature monitoring function and/or the body temperature measurement function through long-range wireless communication. For example, the communication circuit 220 may support long-range wireless communication network (for example, a long-range communication network such as a legacy cellular network, a 5G network, a next-generation communication network, Internet, or a computer network (for example, a local area network (LAN) or a wide area network (WAN))) connection between the electronic device 200 and the server, and may transmit a body temperature measurement result and/or a body temperature monitoring result of the electronic device 200 to the server and store the same in the server, or may receive information on a body temperature measurement history and/or a body temperature monitoring history of a user of the electronic device 200 from the server. For example, the communication circuit 220 may communicate with an external electronic device (for example, the electronic device 104 of FIG. 1) through long-range wireless communication. For example, the communication circuit 220 may support long-range wireless communication network (for example, a long-range communication network such as a legacy cellular network, a 5G network, a next-generation communication network, Internet, or a computer network (for example, a LAN or a WAN)) connection between the electronic device 200 and the external electronic device, and may transmit a body temperature measurement result and/or a body temperature monitoring result of the electronic device 200 to the external electronic device and store the same in the external electronic device, or may display a screen related to body temperature information through a display of the external electronic device or may output a sound or vibration feedback related to body temperature information.

In an embodiment, the communication circuit 220 may support GNSS communication connection between a satellite and the electronic device 200 to position the electronic device 200. The communication circuit 220 may measure a position of the electronic device 200 (for example, the smartphone 201 or the smart watch 202 of FIG. 3) through GNSS communication connection, and may transmit measured position information to the processor 210 or may store the same in the memory 240. For example, the processor 210 may use information on the position of the electronic device 200 or weather or external temperature corresponding to the position as measurement context information.

In an embodiment, the sensor module 230 may include at least one sensor.

For example, the sensor module 230 may include a biometric sensor (for example, a body temperature sensor, a photoplethysmogram (PPG) sensor, an infrared ray (IR) sensor, an electrocardiogram (ECG) sensor, an electrodermal activity (EDA) sensor, or a bioelectrical impedance analysis (BIA) sensor) for detecting biometric data of a user. In another example, the sensor module 230 may include a motion sensor (for example, a gyro sensor, an acceleration sensor, or a proximity sensor) to detect a motion of a user. In still another example, the sensor module 230 may include a sensor (for example, a temperature sensor, a humidity sensor, or an altitude sensor (or an atmospheric pressure sensor)) to detect an external environment or a device use state (for example, temperature, humidity, or altitude) of the electronic device 200.

In an embodiment, the output module 250 may include one or more modules to provide a user interface. For example, the output module 250 may include one or more of a display (for example, the display module 160 of FIG. 1), an audio circuit (for example, the audio module 170 of FIG. 1), a sound output circuit (for example, the sound output module 155 of FIG. 1), and a haptic circuit (for example, the haptic module 179 of FIG. 1).

In an embodiment, the processor 210 of the electronic device 200 may detect a plurality of electronic devices with which a user is in contact or which a user is wearing through the communication circuit 220 and/or the sensor module 230. For example, when a user is using (gripping or is in contact with) the smartphone 201 of FIG. 3 and is wearing the smart watch 202 and the earbud 203 of FIG. 3, the electronic device 200 (for example, one of the smartphone 201 or the smart watch 202) may detect all of the plurality of electronic devices (for example, the smartphone 201, the smart watch 202, and the earbud 203) with which the user is in contact or which the user is wearing. For example, the processor 210 may detect electronic devices which the user is wearing/with which the user is in contact by using a wearing/contact sensing technique which is based on a PPG sensor (or an IR sensor) or an ECG sensor in the sensor module 230. Each electronic device may collect body temperature data (body temperature value and/or skin temperature value) independently through its own body temperature sensor. Body temperature data (body temperature values and/or skin temperature values) collected from the respective electronic devices may be stored and accumulated in a database. For example, the database may correspond to a database in the memory 240 of the electronic device 200 or a database stored in the server 108 of FIG. 1 or interlocked with the server 108.

In an embodiment, the processor 210 of the electronic device 200 may acquire body temperature data (body temperature values and/or skin temperature values) of respective devices from the plurality of electronic devices through the communication circuit 220 and/or the sensor module 230. The body temperature data of respective devices may include at least one of first body temperature data (a first body temperature value and/or a first skin temperature value) which is detected through at least one sensor (for example, a body temperature sensor) of the sensor module 230, and/or second body temperature data (a second body temperature value and/or a second skin temperature value) received through the communication circuit 220.

For example, the processor 210 of the electronic device 200 (for example, the smartphone 201 of FIG. 3) may detect a skin temperature value by itself through the sensor module 230 (for example, a body temperature sensor), or may measure a body temperature value by using the detected skin temperature value. In another example, the processor 210 of the electronic device 200 (for example, the smartphone 201 of FIG. 3) may receive body temperature data (a body temperature value and/or a skin temperature value) from an external electronic device (for example, the smart watch 202 or the earbud 203) through the communication circuit 220 (for example, short-range wireless communication connection). In still another example, the electronic device 200 (for example, the smartphone 201 of FIG. 3) may calculate a body temperature value based on a skin temperature value received from an external electronic device (for example, the smart watch 202 or the earbud 203).

In an embodiment, the body temperature sensor may detect a skin temperature and/or a body temperature of a user. The skin temperature may refer to a temperature on a skin surface where the body temperature sensor is positioned. A body temperature may be estimated (or calculated) from the skin temperature. The skin temperature and/or the body temperature estimated from the skin temperature may be influenced by a measurement context.

In an embodiment, the processor 210 may determine a representative electronic device among multiple electronic devices with which a user is in contact or which a user is wearing, based on measurement context information. The representative electronic device may be a device that has a relatively higher correlation with real body temperature than other electronic devices, or has relatively lower sensitivity to a measurement context than other electronic devices among the multiple electronic devices. The representative electronic device may correspond to a device that is less influenced by a measurement context and is able to measure a body temperature relatively more accurately than other electronic devices among the multiple electronic devices.

In an embodiment, the measurement context information may include at least one of information on an external environment (for example, an external temperature, humidity, altitude), information on a user state (for example, a static state, a dynamic state), information on a device use state (for example, a device using time, a device internal temperature), and information on a device characteristic (for example, a measuring area, a device type, a sensor arrangement structure).

In an embodiment, the processor 210 may identify the measurement context information to determine the representative electronic device. For example, the measurement context information may be information on at least a part of an external temperature, a device using time or a device internal temperature of each of multiple electronic devices with which a user is in contact or which a user is using, a user state, and a device type, a measuring area, or a sensor arrangement structure of each of the multiple electronic devices.

In an embodiment, the measurement context information may include information which is acquired through the sensor module 230 and/or the communication circuit 220, and/or information which is pre-stored in the memory 240. The measurement context information may be collected in various ways. For example, at least part (for example, an external temperature, humidity, altitude, a device internal temperature) of the measurement context information may be detected in real time through at least one sensor. In another example, at least part (for example, a user state, a device using time) of the measurement context information may be processed and/or calculated by using information that is detected through at least one sensor. In still another example, at least part (for example, a measuring area, a device type, a sensor arrangement structure) of the measurement context information may be pre-stored.

In an embodiment, the processor 210 of the electronic device 200 may identify an external temperature.

For example, the processor 210 may measure an external temperature (or an ambient temperature) by using its own temperature sensor.

In another example, the processor 210 may receive information on weather or external temperature corresponding to its position from an external electronic device (for example, the server 108 of FIG. 1) through the communication circuit 220.

In still another example, the processor 210 may identify a temperature of a current place by communicating with a surrounding external electronic device (for example, a surrounding IoT device such as an air conditioner) through the communication circuit 220.

In yet another example, the processor 210 may estimate an external temperature based on body temperature values (or skin temperature values) of respective devices which are measured through a plurality of electronic devices which the user is wearing or with which the user is in contact. For example, if a difference between a first body temperature value measured in the earbud 203 worn on the ears and a second body temperature value measured in the smart ring 204 worn on the finger is less than a designated offset value, it may be estimated that the external temperature is substantially analogous to user's body temperature. If the first body temperature value is greater than the second body temperature value and the difference between the first body temperature value and the second body temperature value is greater than or equal to the offset value, it may be estimated that the external temperature falls within a low-temperature range that influences measurement of user's body temperature. If the second body temperature value is greater than the first body temperature value and the difference between the first body temperature value and the second body temperature value is greater than or equal to the offset value, it may be estimated that the external temperature falls within a high-temperature range that influences measurement of user's body temperature.

In an embodiment, the processor 210 may identify a device using time (for example, a device wearing or contact time) of each electronic device. For example, the processor 210 may identify a device using time of a wearable electronic device (for example, the smart watch 202 of FIG. 3) in a wearing/contact detection technique using a PPG sensor (or an IR sensor) or an ECG sensor in the sensor module 230. In another example, the processor 210 may identify a device using time of an electronic device (for example, the smartphone 201 of FIG. 3) based on a power consumption level or a duration for which the output module 250 (for example, a display) is maintained in an active state.

In an embodiment, the processor 210 may identify a device internal temperature of each electronic device. For example, the processor 210 may measure its own device internal temperature through a temperature sensor in the sensor module 230, or may receive a device internal temperature of another electronic device which the user is wearing or with which the user is in contact through the communication circuit 220.

In an embodiment, the processor 210 of the electronic device 200 may identify a measuring area of each device. For example, the measuring area of each device may be any one of the wrist, ankle, arm, leg, ear, finger, toe or eye. The measuring area of each device may correspond to a device type (or a device identification (ID)). For example, information on measuring areas of respective devices may be pre-stored, and the measuring area of each device may be identified based on the stored information. For example, the measuring area (or contact area) of the smartphone 201 may be the palm or finger. The measuring area (or wearing area) of the smart watch 202 may be the wrist. The measuring area of the earbud 203 may be the ears. The measuring area of the smart ring 204 may be the finger. The measuring area of the smart band 207 may be the wrist or ankle.

In an embodiment, a correlation with real body temperature or sensitivity to a measurement context in each of the multiple electronic devices may be a criterion for selecting a representative electronic device.

The correlation with real body temperature may be related to a mechanical characteristic (for example, at least one of a measuring area, a sensor arrangement structure or a device type). For example, a device that is worn on the ears having many blood vessels and close to the heart may have a relatively higher correlation with real body temperature than other electronic devices. The correlation with real body temperature may be relatively lower toward the distal body parts (for example, the finger) that are farther away from the heart.

The sensitivity to the measurement context (for example, an external temperature, a device wearing time, a user state) is a degree of influence of the measurement context when a body temperature is measured, and may be related with a mechanical characteristic (for example, at least one of a measuring area, a sensor arrangement structure, or a device type).

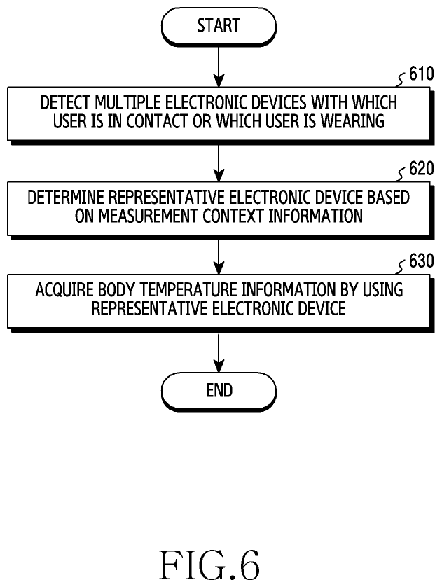

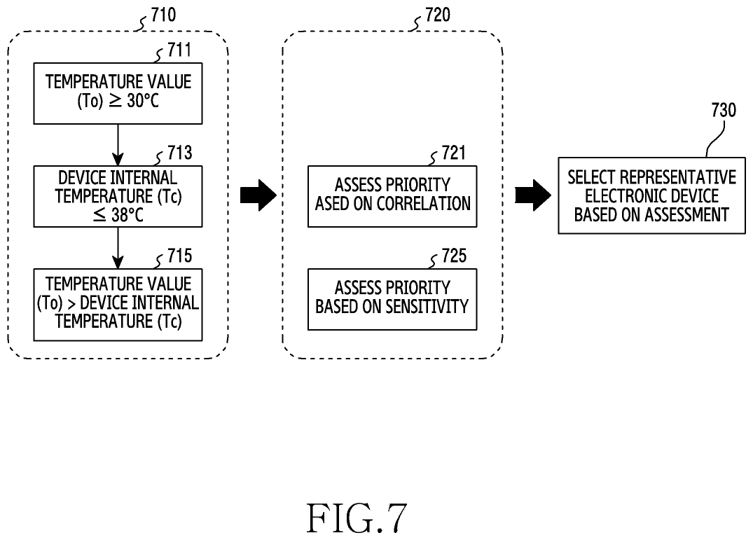

The correlation with real body temperature and the sensitivity to the measurement context will be described in detail below with reference to FIGS. 6, 7, and Table 1.

In an embodiment, the electronic device 200 (for example, the memory 240 of the electronic device 200) may pre-store correlation information indicating a correlation with real body temperature for each of a plurality of electronic devices, and sensitivity information indicating sensitivity to a measurement context. Information on a correlation level with real body temperature may be stored for each device. Sensitivity to a measurement context may be different for each measurement context factor, each device. For example, the measurement context factor may correspond to at least one of an external temperature, a device using time, a device internal temperature, a device type, a sensor arrangement structure, a measuring area, or a user state. For example, information on a sensitivity level of each device to an external temperature, a sensitivity level of each device to a device using time, and/or a sensitivity level of each device to a user state may be pre-stored (see Table 1).

In an embodiment, the processor 210 may determine a representative electronic device, based on at least part of the measurement context information, correlation information indicating a correlation with real body temperature, and sensitivity information indicating sensitivity to a measurement context.

In an embodiment, the processor 210 may select a representative electronic device from the plurality of electronic devices which the user is wearing or with which the user is in contact, based on a user state of the measurement context information.

A method of selecting a representative electronic device based on a user state is as follows.

For example, a user state may be any one of a static state and a dynamic state. The dynamic state may be a state (for example, a kinetic state) in which a change in user's body temperature is greater than or equal to a predetermined offset value, a user is sweating, or a motion is detected for a predetermined time or longer. The static state may be a state except for the dynamic state or a state in which a change in user's body temperature is maintained within a predetermined range (for example, a sitting or lying state, or a sleep state). For example, the electronic device 200 may determine whether the user state changes from the static state to the dynamic state, based on sensing information (for example, body temperature, motion, humidity) which is detected in at least one of the plurality of electronic devices which the user is wearing or with which the user is in contact.

In an embodiment, when the user state is the static state, the representative electronic device may be selected based on the correlation information of the plurality of electronic devices indicating a correlation with real body temperature. When the user state is the dynamic state, the representative electronic device may be selected based on the sensitivity information of the plurality of electronic devices indicating sensitivity to a measurement context.

In an embodiment, the processor 210 may select a representative electronic device from the plurality of electronic devices which the user is wearing or with which the user is in contact, based on an external temperature of the measurement context information.

An example of a method of selecting a representative electronic device based on an external temperature is as follows.

For example, when the external temperature is a first level (for example, about 15° C. to 25° C., a room temperature range that does not influence body temperature measurement), the processor 210 may select, as a representative electronic device, a device that has the highest correlation level from the plurality of electronic devices with which the user is in contact or which the user is wearing, with reference to a correlation with real body temperature. For example, when the external temperature is the first level, the earbud 203 that has the highest correlation level with real body temperature may be selected as a representative electronic device from the earbud 203, the smart watch 202, and the smart ring 204 that the user is wearing.

When the external temperature is a second level (for example, less than about 15° C., a low temperature range that influences body temperature measurement), the processor 210 may select, as a representative electronic device, a device that has the lowest sensitivity level to the external temperature from the plurality of electronic devices with which the user is in contact or which the user is wearing, with reference to sensitivity to the external temperature. For example, when the external temperature is the second level, the earbud 203 that has the lowest sensitivity level to the external temperature may be selected as a representative electronic device from the earbud 203, the smart watch 202, and the smart ring 204 that the user is wearing.

When the external temperature is a third level (for example, about 30° C. or higher, a high temperature range that influences body temperature measurement), the processor 210 may select a representative electronic device with reference to sensitivity to a user state. When the external temperature is the third level, the human body may achieve thermal equilibrium by increasing the total heat loss by increasing an in-body heat storage rate or through sweat evaporation, and may maintain body temperature. Due to such thermoregulation of the human body, the user state may change from the static state to the dynamic state, and the dynamic statue of the user may influence body temperature measurement. For example, when the external temperature is the third level, the smart ring 204 that has the lowest sensitivity level to the user state may be selected as a representative electronic device from the earbud 203, the smart watch 202, and the smart ring 204 that the user is wearing.

In an embodiment, the processor 210 of the electronic device 200 may acquire body temperature data of respective devices from the plurality of electronic devices through the communication circuit 220 and/or the sensor module 230.

The body temperature of the respective devices may include body temperature values of the respective devices (and/or skin temperature values of the respective devices) which are measured by the plurality of electronic devices with which the user is in contact or which the user is wearing. The body temperature values of the respective devices may further include device information (for example, a device ID) on the electronic devices which measure respective body temperature values (and/or skin temperature values).

For example, the plurality of electronic devices which provide body temperature values (or skin temperature values) may be the electronic device 200 (for example, the smartphone 201 of FIG. 3) and one or more external electronic devices (for example, the smart watch 202, the earbud 203 of FIG. 3). The one or more external electronic devices may transmit their own device information (for example, device IDs) and their measured body temperature values (or skin temperature values) to the electronic device 200 through the communication circuit 220 (or short-range wireless communication connection).

In another example, the plurality of electronic devices which provide body temperature values (or skin temperature values) may be two or more external electronic devices (for example, the smart watch 202, the smart band 207). The two or more external electronic devices may transmit their own device information (for example, device IDs) and their measured body temperature values (or skin temperature values) to the electronic device 200 through the communication circuit 220 (or short-range wireless communication connection).

In an embodiment, the processor 210 may identify one or more valid electronic devices among the plurality of electronic devices, based on whether temperature data of each device satisfies a designated condition.

In an embodiment, when a condition in which a body temperature value measured in a first electronic device among the plurality of electronic devices is greater than or equal to a first threshold (for example, about 30° C.), a condition in which a device internal temperature of the first electronic device is less than or equal to a designated second threshold (for example, about 38° C.), and a condition in which the body temperature value exceeds the device internal temperature are satisfied, the processor 210 may identify the first electronic device as a valid electronic device. A method of identifying a valid electronic device will be described in detail below with reference to FIG. 7.

In an embodiment, the processor 210 may assess the priority of each of one or more valid electronic devices, based on measurement context information. The processor 210 may select a representative electronic device from the one or more valid electronic devices, based on a result of assessing the priority.

A method of assessing a priority based on an external temperature and a user state of the measurement context information is as follows.

For example, when the user state is the static state and the external temperature is the first level (for example, a room temperature range from about 15° C. to 25° C.) that does not influence body temperature measurement, the highest priority may be assigned to the earbud 203 that has the highest correlation with real body temperature among the smart watch 202, the earbud 203, and the smart ring 204 that the user is wearing, considering the wearing area (for example, the finger, wrist, ear) of each device. The earbud 203 assigned the highest priority may be selected as a representative electronic device.

When the user state is the static state and the external temperature is the second level (for example, less than about 15° C., a low temperature range that influences body temperature measurement), the highest priority may be assigned to the earbud 203 that has the lowest sensitivity to the external temperature among the smart watch 202, the earbud 203, and the smart ring 204 that the user is wearing, considering the wearing area of each device and/or the sensor arrangement structure (for example, an open type, a semi-open type, a sealed type). The earbud 203 assigned the highest priority may be selected as a representative electronic device.

When the user state is the dynamic state (for example, when body temperature actively changes and/or when sweat is produced) or the external temperature is the third level (for example, about 30° C. or higher, a high temperature range that influences body temperature measurement), the highest priority may be assigned to the smart ring 204 that has the lowest sensitivity to the user state among the smart watch 202, the earbud 203, and the smart ring 204 that the user is wearing, considering the wearing area of each device and/or the sensor arrangement structure (for example, an open type, a semi-open type, a sealed type). The smart ring 204 assigned the highest priority may be selected as a representative electronic device.

In an embodiment, the processor 210 of the electronic device 200 may select a representative electronic device that is able to provide body temperature information relatively more accurately than other electronic devices among the multiple electronic devices with which the user is in contact or which the user is wearing according to a measurement context. The representative electronic device may be maintained or changed according to a real-time measurement context. Through the representative electronic device that has a relatively higher correlation with real body temperature than other electronic devices, or is relatively less influenced by a measurement context than other electronic devices, relatively more accurate body temperature information (a skin temperature value and a body temperature value estimated based on the skin temperature) may be provided.

In an embodiment, the processor 210 may refine (or redetermine) the representative electronic device in response to at least one of a first event which occurs as a designated time comes, a second event which occurs as the user is in contact with or is wearing a new electronic device, and a third event which occurs as user's contact with or wearing the electronic device is released.

In an embodiment, the processor 210 of the electronic device 200 may acquire body temperature information of the user by using the representative electronic device. The body temperature information may correspond to information on body temperature and/or skin temperature. The processor 210 may provide a body temperature measuring function and/or a body temperature monitoring function by using the acquired body temperature information.

In an embodiment, the processor 210 of the electronic device 200 may provide (or output) a user interface related to the body temperature information acquired by using the representative electronic device.

For example, the processor 210 of the electronic device 200 (for example, the smartphone 201 of FIG. 3) may output the user interface (for example, at least part of a screen, a voice, and a vibration) related to the body temperature information through the output module 250 of the electronic device 200. In another example, the processor 210 of the electronic device 200 (for example, the smartphone 201 of FIG. 3) may transmit information on the user interface related to the body temperature information to one or more external electronic devices (for example, the smart watch 202) through the communication circuit 220 to cause the one or more external electronic devices to output the user interface (for example, at least part of a screen, a voice, and a vibration).

In an embodiment, when there is no valid electronic device that satisfies the designated condition, the processor 210 may determine a cause (for example, any one of an external temperature, a device using time, a device internal temperature, a charging state or a user dynamic state) of a body temperature measurement error, based on the measurement context information. The processor 210 may provide a user interface including information showing the cause of the body temperature measurement error through the output module 250.

In an embodiment, the processor 210 may determine one or more representative electronic devices with respect to a plurality of time sections. The processor 210 may acquire body temperature information measured by the one or more representative electronic devices, and may provide a user interface related to the body temperature information through the output module 250.

FIG. 3 is a view illustrating use states of multiple electronic devices according to an embodiment of the disclosure.

An electronic device (for example, the electronic device 200 of FIG. 2) according to an embodiment may be any one of the smartphone 201, the smart watch 202, the earbud 203, the smart ring 204, the smart glasses 205, the smart patch 206, or the smart band 207.

In an embodiment, the plurality of electronic devices (for example, two or more of the smartphone 201, the smart watch 202, the earbud 203, the smart ring 204, the smart glasses 205, the smart patch 206, or the smart band 207) may perform a body temperature measurement function. The electronic device 200 (for example, the smartphone 201 or the smart watch 202) may perform a body temperature monitoring function. The electronic device 200 may selectively perform only one of the two functions of the body temperature monitoring function and the body temperature measurement function, and may perform both of the two functions.

For example, the smartphone 201 may perform the body temperature monitoring function, and one or more wearable electronic devices may perform the body temperature measurement function. The one or more wearable electronic devices may correspond to one or more of the smart watch 202, the earbud 203, the smart ring 204, the smart glasses 205, the smart patch 206, or the smart band 207. In this case, the smartphone 201 may collect body temperature data of respective devices (body temperature values and/or skin temperature values) from the one or more wearable electronic devices. The smartphone 201 may measure body temperature (or skin temperature) by itself through at least one sensor (for example, a body temperature sensor) provided therein.

In an embodiment, body temperature measurement and/or body temperature monitoring may be performed by using two or more electronic devices that the user is using, touching, attaching, or wearing.

Referring to FIG. 3, the plurality of electronic devices may have various measuring areas (for example, wearing areas or contact areas). For example, body temperature values (or skin temperature values) may be collected by measuring body temperature in one or more electronic devices of the smart watch 202 worn on the wrist, the earbud 203 worn on the ears, the smart ring 204 worn on the finger, the smart glasses 205 worn on the eyes, the smart patch 206 attached to a part of the body, or the smart band 207 worn on the wrist or ankle. For example, the measuring area may be any one of the wrist, ankle, arm, leg, ear, finger, toe, or eye.

In an embodiment, each electronic device 200 may estimate (or calculate) a body temperature from a skin temperature. For example, each electronic device 200 may detect a skin temperature through at least one sensor (for example, a contact type or non-contact type body temperature sensor), and may convert the skin temperature into a body temperature by using a designated regression equation (or regression model). A skin temperature of a part of the body may be less influenced by a measurement context than skin temperature of other body parts. Substantially accurate body temperature may be measured from a skin temperature which is less influenced by a measurement context. When the skin temperature is greatly influenced by the measurement context, a body temperature estimated from the corresponding skin temperature may be substantially inaccurate. For example, when an external temperature is a designated level (for example, a low temperature range of less than about 15° C.), a device internal temperature is higher than a threshold (for example, about 38° C.), or a user state is a dynamic state in which body temperature is dramatically increased or sweat is produced, the measurement context may influence a skin temperature and/or body temperature measurement using the skin temperature. Due to the influence of the measurement context, a body temperature estimated from a skin temperature of a part of the body may be relatively inaccurate compared to a body temperature estimated from a skin temperature of other body parts.

According to an embodiment, a representative electronic device that is able to measure a body temperature relatively more accurately than other electronic devices may be selected from multiple electronic devices according to a measurement context, and may be used, so that relatively more accurate body temperature information may be provided to the user. Since mechanical characteristics (for example, a measuring area, a device type or a sensor arrangement structure) of the plurality of electronic devices are different and skin temperatures of respective body parts are different, regression models for converting skin temperatures detected in the electronic devices into body temperatures may be different from one another.

For example, measuring areas (or measuring positions) of the smartphone 201, the smart watch 202, and the earbud 203 may be the finger, wrist, and inside of the ear, respectively. Therefore, body temperature sensors provided in the smartphone 201, the smart watch 202, and the earbud 203, respectively, may apply different parameter values for converting skin temperature into body temperature.

For example, the smartphone 201, the smart watch 202, and the earbud 203 may detect skin temperatures, and may estimate (or calculate) body temperature values from the detected skin temperatures by using Equation 1. A body temperature value may be a value resulting from calibration or tuning of a skin temperature detected from a part of the body.

Body temperature=a*(skin temperature)+b (a,b: regression coefficients) Equation 1

The smartphone 201, the smart watch 202, and the earbud 203 may have different mechanical characteristics (for example, a measuring area, a sensor arrangement structure, or a device type), and thus, may have different regression coefficient values (values of a, b). For example, since skin temperature varies according to measuring areas although user's body temperature (core temperature or representative temperature) is uniform, electronic devices may have different regression coefficient values for converting skin temperature into body temperature according to measuring areas of devices. Regression coefficients for calculating body temperatures of respective devices may be pre-stored. The regression coefficients for calculating body temperatures of respective devices may be obtained from experimental data.

Equation 1 presented above is just an example for easy understanding, and a method of calculating a body temperature of each device is not limited thereto and may be modified, applied, or extended in various ways.

A skin temperature detected in each electronic device 200 and/or a body temperature estimated from the skin temperature may be influenced by a measurement context (for example, at least one of a measuring area, an external temperature, a device using time, a device internal temperature, a device type, a sensor arrangement structure or a user state). According to the measurement context, a skin temperature which is greatly influenced by the measurement context may be detected in a part of multiple electronic devices. A result of measuring body temperature by using the skin temperature which is greatly influenced by the measurement context may be substantially inaccurate. Even if user's real body temperature is uniformly maintained, user's skin temperature detected in each of the multiple electronic devices and/or the body temperature measurement result may be different according to a degree of influence of the measurement context. The electronic device that is capable of measuring body temperature relatively accurately may vary according to a real-time measurement context.

According to an embodiment, when the user is using (for example, touching or wearing) the plurality of electronic devices (for example, the smartphone 201, the smart watch 202 and the earbud 203), the electronic device 200 (for example, the smartphone 201 or the smart watch 202) may determine a representative electronic device (for example, any one of the smartphone 201, the smart watch 202 and the earbud 203) among the plurality of electronic devices, based on measurement context information. The electronic device 200 may provide a body temperature measurement function and/or a body temperature monitoring function by using the determined representative electronic device. The representative electronic device may be a device that is capable of measuring body temperature relatively more accurately than other electronic devices among multiple electronic devices that the user is using. The representative electronic device may be a device that provides body temperature information having the highest correlation with real body temperature or provides body temperature information least influenced by a measurement environment among the multiple electronic devices. The electronic device 200 may select a representative electronic device according to a real-time measurement context, and may receive body temperature information which is relatively accurate compared to that of other electronic devices from the representative electronic device, and may provide the body temperature information to the user.

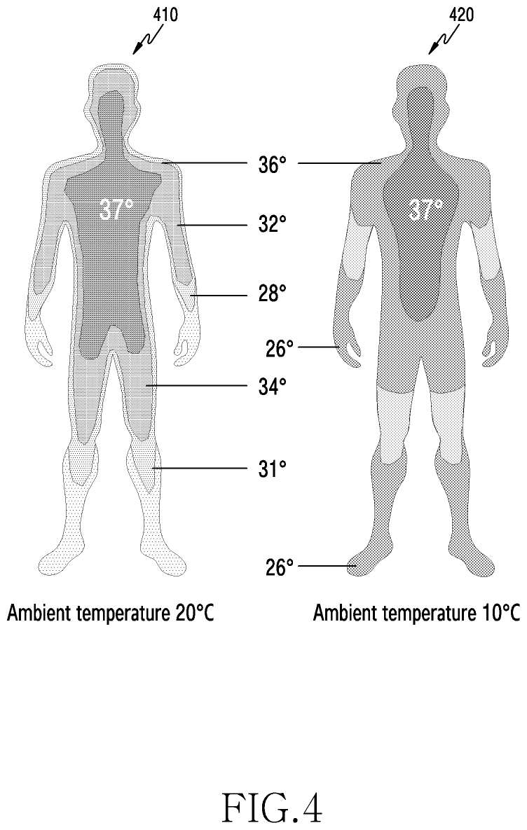

FIG. 4 is a view illustrating temperature differences by body parts according to external temperatures to explain an operation method of an electronic device according to an embodiment of the disclosure.

Reference numeral 410 of FIG. 4 illustrates temperature distribution by body parts when an external temperature is a first level (for example, ambient temperature of about 20° C., a room temperature range). Reference numeral 420 illustrates temperature distribution by body parts when the external temperature is a second level (for example, ambient temperature of about 10° C., a low-temperature range).

As shown in reference numeral 410, there may be a temperature difference according to body parts. The human body may have a highest temperature (core temperature, for example, about 37° C.) in the center of the body, and may have lower skin temperatures (for example, about 27 to 28° C.) toward distal body parts (finger, toe) thar are farther away from the center of the body. The temperature of a distal body part may be relatively lower than that of the center.

The temperature of each body part may be influenced by an external temperature. The degree of influence of the external temperature (sensitivity to the external temperature) may vary according to body parts. As the sensitivity to the external temperature is higher, the influence of the external temperature may be greater. A distal body part (for example, wrist or finger) whose skin is exposed to the outside may be relatively more sensitive to the external temperature than the center of the body. On the other hand, a body part whose skin is not exposed to the outside or is less exposed may be relatively less sensitive to the external temperature than distal body parts.

In addition, the degree of influence of an external temperature on temperatures of various body parts may vary according to which body part it is and/or what the external temperature is.

As shown in reference numeral 420, when the user is exposed to the second level (for example, ambient temperature of about 10° C.) which is a relatively low external temperature, the influence of the external temperature may be greater than when the external temperature is the first level (for example, about 20° C.). Accordingly, a temperature deviation between the center of the body and distal body parts (for example, two arms, two legs) may become greater. As a body part is farther away from the center of the body, the degree of exposure of the body part to the outside may increase and a skin temperature may more dramatically decrease. The influence of the external temperature may be greater toward distal body parts or as the degree of exposure to the outside is greater. For example, the degree of decrease of temperature (for example, about 26° C.) may be greater in the finger that is more exposed to the outside than in the inside of the ears.

As described above, the external temperature may influence the temperature of each body part and a body temperature measurement result using the same. The influence of the external temperature and the sensitivity to the external temperature may vary according to a measuring area.

According to an embodiment, when a body temperature is measured, highly accurate body temperature information may be selectively provided among body temperature values (or skin temperature values) of respective devices which are measured by using multiple electronic devices positioned in respective body parts, considering a measurement context such as an external temperature and/or a measuring area.

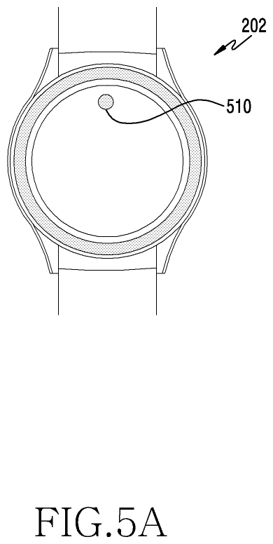

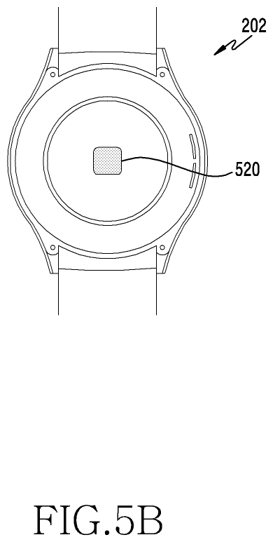

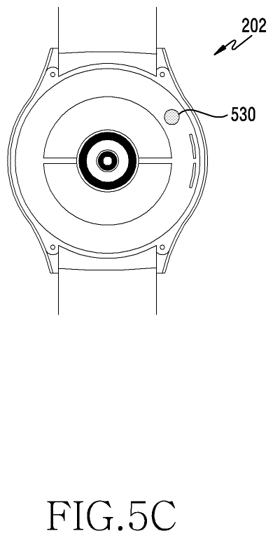

FIGS. 5A, 5B, and 5C are views to explain sensor types and sensor arrangement structures which are applicable to an electronic device according to various embodiments of the disclosure.

In various embodiments, a body temperature sensor may be a contact type temperature sensor or a non-contact type temperature sensor.

The contact type temperature sensor may measure a temperature in direct contact with a target for measuring. For example, the contact type temperature sensor may be any one of a thermocouple sensor which detects an electromotive force of a specific temperature, a resistance temperature detector which detects a resistance changing with temperature, or a thermistor.

The non-contact type temperature sensor may measure a temperature with infrared ray emissivity of a measuring target without contacting the measuring target. For example, the non-contact type temperature sensor may be a thermopile infrared sensor. The corresponding sensor may be configured to include a lens for focusing infrared energy. The corresponding sensor may perform compensation processing with respect to a change in ambient temperature, and then, may convert collected energy into an electrical signal which is displayable in a temperature unit. The non-contact type temperature sensor may be typically used in a place where a contact type temperature sensor is not allowed. When the contact type temperature sensor is used, it may be possible to directly detect a skin temperature and to measure a body temperature by using the skin temperature. When a rapid reaction time is required, the non-contact type temperature sensor may be used.

Types of electronic devices including a contact type and/or non-contact type temperature sensor and providing a body temperature measuring function are illustrated in FIG. 3.

According to an embodiment, a sensor type and/or a sensor arrangement structure for body temperature measurement may be determined according to mechanical characteristics (for example, a device type or a measuring area) of the electronic device (for example, any one of the smartphone 201, the smart watch 202, the earbud 203, the smart ring 204, the smart glasses 205, the smart patch 206 or the smart band 207 of FIG. 3). For example, the device type may be any one of a draw-in type, a watch type, a ring type, a phone type (or contact type), a glasses type, a band type, and an attachment type. The sensor type may be any one of a contact type or a non-contact type. The sensor arrangement structure may be any one of an open type, a semi-open type and a sealed type.

For example, a body temperature sensor of a smartphone (for example, the smartphone 201 of FIG. 3) may be a non-contact type temperature sensor. Since the smartphone (for example, the smartphone 201 of FIG. 3) is not continuously used (touched or gripped), the smartphone may need to measure a body temperature in an on-spot check method. A non-contact type temperature sensor that has a rapid reaction time may be used to perform on-spot check.

In another example, since a smart patch (for example, the smart patch 206 of FIG. 3) is usually attached to user's skin and is mainly used for long-term monitoring, a contact type body temperature sensor may be used for the smart patch (for example, the smart patch 206 of FIG. 3).

In still another example, since an earbud (for example, the earbud 203 of FIG. 3) is structured to be worn on the inside of user's ears, the earbud may use a non-contact type temperature sensor like a tympanic thermometer which is a dedicated thermometer, and may have a sensor arrangement structure of a sealed type.

In yet another example, smart glasses (for example, the smart glasses 205 of FIG. 3) may use both a contact type temperature sensor and a non-contact type temperature sensor according to a body temperature measuring area. The smart glasses (for example, the smart glasses 205 of FIG. 3) are mostly worn for a long time and have a mechanical characteristic of being robustly fixed relative to user's motion. The smart glasses may have both a contact area and a non-contact area due to its mechanical characteristics. When a temperature sensor is disposed in a temple tip of glasses contacting the ear or in a nose pad contacting the nose, a contact type temperature sensor may be used as the body temperature sensor. When a body temperature sensor is disposed in a temple of glasses to measure a body temperature on user's temple, which is a non-contact body part, a non-contact type temperature sensor may be used as the body temperature sensor.

In a further example, since a smart ring (for example, the smart ring 204 of FIG. 3) is able to continuously measure in contact with the finger, it may be efficient to use a contact type temperature sensor to be able to continuously collect body temperature information in a contact state.

In a further additional example, a smart watch (for example, the smart watch 202 of FIG. 3) or a smart band (for example, the smart band 207 of FIG. 3) may use both a contact type temperature sensor and a non-contact type temperature sensor according to a sensor arrangement structure (or a measuring area) of a body temperature sensor or a mechanical structure.

For example, if the body temperature sensor is positioned on the front surface of a device that does not contact the skin, it may be efficient to use a non-contact type temperature sensor for collecting body temperature information. If the body temperature sensor is positioned on the center of the rear surface of a device that directly contacts the wrist skin, it may be efficient to use a contact type temperature sensor for continuously collecting body temperature information. If it is difficult to place a body temperature sensor on the center of the rear surface of the device since another sensor (for example, an ECG sensor or a PPG sensor) is disposed in the center of the rear surface of the device, a non-contact type body temperature sensor may be disposed on a side of the rear surface and may be used as the body temperature sensor.

FIGS. 5A, 5B, and 5C illustrate sensor types and/or sensor arrangement structures when the electronic device (for example, the electronic device 200 of FIG. 2) is a smart watch (for example, the smart watch 202 of FIG. 3) which is a wearable device according to various embodiments of the disclosure. The smart watch (for example, the smart watch 202 of FIG. 3) may include a body temperature sensor. The body temperature sensor may include a non-contact type temperature sensor 510, 530, and/or a contact type temperature sensor 520.

Referring to FIG. 5A, the smart watch 202 may include the non-contact type temperature sensor 510 positioned on the front surface of the device. Since the front surface of the device does not directly contact the skin due to the mechanical characteristics of the smart watch 202, it may be efficient to arrange the non-contact type temperature sensor 510 as the body temperature sensor for collecting body temperature information.

Referring to FIG. 5B, the smart watch 202 may include the contact type temperature sensor 520 positioned on the center of the rear surface of the device. Since the rear surface of the device is in contact with the skin for a relatively long time due to the mechanical characteristics of the smart watch 202, it may be efficient to arrange the contact type temperature sensor 520 as the body temperature sensor for continuously collecting body temperature information.

When there is another sensor (for example, an ECG sensor or a PPG sensor) on the center of the rear surface of the smart watch 202, the non-contact type temperature sensor 530 may be arranged on a side (for example, an outside) of the rear surface that does not contact the skin in order to avoid interference and to implement an efficient structure necessary for collecting body temperature information, as shown in FIG. 5C, and may be used as the body temperature sensor.

In the above-described examples, various sensor arrangement structures related to the sensor types (contact type and non-contact type) have been described, but the sensor arrangement structure may be variously modified or utilized according to mechanical characteristics (for example, a device type or a device structure).

FIG. 6 is a flowchart illustrating an operation method of an electronic device according to an embodiment of the disclosure.

For the convenience of explanation, it is assumed that the method of FIG. 6 is performed by the electronic device 200 of FIG. 2. However, this should not be considered as limiting. For example, the method of FIG. 6 may be performed by the processor 210 of the electronic device 200, an application (for example, a body temperature application, a health care application) executed in the electronic device 200, or the electronic device 101 or the processor 120 of FIG. 1. In a certain embodiment, at least one of operations of the method illustrated in FIG. 6 may be omitted, the sequence of some operations may be changed, or other operations may be added.

In an embodiment, a user may be in contact with or may be wearing a plurality of electronic devices (for example, at least part of the smartphone 201, the smart watch 202, the earbud 203, the smart ring 204, the smart glasses 205, the smart patch 206, the smart band 207 of FIG. 3). One (for example, the smart phone 201) of the plurality of electronic devices with which the user is in contact with or which the user is wearing may perform a body temperature monitoring function and/or a body temperature measurement function. The other devices (for example, at least part of the smart watch 202, the earbud 203, the smart ring 204, the smart glasses 205, the smart patch 206, the smart band 207) of the plurality of electronic devices may perform the body temperature measurement function.

In operation 610, the electronic device 200 may detect the plurality of electronic devices with which the user is in touch or which the user is wearing. For example, when the user is using (gripping or touching) the smartphone 201 and is also wearing the smart watch 202 and the earbud 203, the electronic device 200 (for example, the smartphone 201) may detect all of the plurality of electronic devices (for example, the smartphone 201, the smart watch 202, and the earbud 203) with which the user is in contact or which the user is wearing. For example, the electronic device 200 may detect the electronic devices that the user is wearing/touching by using a wearing/contact detection technique based on a PPG sensor (or an IR sensor) or an ECG sensor. Each electronic device may measure a body temperature value (or a skin temperature value) independently through its own body temperature sensor. Body temperature values (or skin temperature values) collected from the respective electronic devices may be stored and accumulated in a database.

In operation 620, the electronic device 200 may determine a representative electronic device among the plurality of electronic devices with which the user is in contact or which the user is wearing, based on measurement context information.

In an embodiment, the measurement context information may include at least one of information on an external environment (for example, an external temperature, humidity, altitude), information on a user state (for example, a static state, a dynamic state), information on a device use state (for example, a device using time, a device internal temperature), and information on device characteristics (for example, a measuring area, a device type, a sensor arrangement structure).

In an embodiment, the electronic device 200 may select the representative electronic device considering a correlation with real body temperature of each of the plurality of electronic devices with which the user is in contact or which the user is wearing, and/or sensitivity to a measurement context.

Body parts that are closer to the core of the body may have a higher correlation with real body temperature. The correlation with real body temperature may be related to a mechanical characteristic (for example, at least one of a measuring area, a device type and a sensor arrangement structure). For example, when measuring areas are the ear, wrist, and finger, the ear that has many blood vessels and is close to the heart may have a relatively higher correlation with real body temperature than other measuring areas (for example, the wrist, the finger). The correlation with real body temperature may be lower toward the distal body parts (for example, the finger) that are farther away from the heart. For example, in the case of the smartphone 201 (the device type: phone type, the contact area: finger, the sensor arrangement structure: open type), the smart watch 202 (the device type: watch type, the wearing area: wrist, the sensor arrangement structure: semi-open type), and the earbud 203 (the device type: draw-in type, the wearing area: ear, the sensor arrangement structure: sealed type), the earbud 203 may have a relatively higher correlation with real body temperature than the other electronic devices, and the smartphone 201 may have a relatively lower correlation with real body temperature than the other electronic devices, considering mechanical characteristics (for example, a measuring area, a device type and/or a sensor arrangement structure).

Sensitivity to a measurement context may be understood as a degree of influence of the measurement context when a body temperature is measured. The sensitivity to the measurement context may be related to mechanical characteristics (for example, a measuring area, a device type, or a sensor arrangement structure).

For example, sensitivity of each device to an external temperature may be as follows. When the measuring area (or wearing area) is the ears like the earbud 203, a body temperature sensor may be disposed inside the ears and may have a sensor arrangement structure of a sealed type that is less exposed to the outside, and accordingly, sensitivity to an external temperature may be low. When the measuring area is the finger like the smart ring 204, sensitivity to an external temperature may be high due to the sensor arrangement structure of the open type. When the measuring area is the wrist like the smart watch 202, the sensor arrangement structure does not have a completely sealed structure but has a semi-open type such that a contact surface between the skin and the device is blocked from the outside to some extent, and accordingly, sensitivity to an external temperature may be an intermediate level.

In another example, sensitivity of each device to a user state may be as follows. Sensitivity of the earbud 203 to a user state among the earbud 203, the smart watch 202, and the smart ring 204 may be relatively higher than the other electronic devices, and sensitivity of the smart ring 204 may be relatively lower than the other electronic devices. Since the earbud 203 (the wearing area: ear, the device type: draw-in type, the sensor arrangement structure: sealed type) has a sensor arrangement structure of a sealed type so as to be worn inside the ears, the earbud 203 is less sensitive to an external temperature, but is more sensitive to a user state (for example, a state in which a body temperature is dramatically changed or sweat is produced). Since the smart ring 204 (the wearing area: finger, the device type: ring type, the sensor arrangement structure: open type) has a sensor arrangement structure of an open type, the smart ring may be less sensitive to a user state. Since the smart watch 202 (the wearing area: wrist, the device type: watch type, the sensor arrangement structure: semi-open type) has a sensor arrangement structure of a semi-open type, the smart watch may have sensitivity of an intermediate level to a user state.

In an embodiment, the electronic device 200 (for example, the memory 240 of the electronic device 200) may pre-store correlation information indicating a correlation with real body temperature for each of the plurality of electronic devices, and sensitivity information indicating sensitivity to a measurement context. Information on a correlation level with real body temperature may be stored for each device. Sensitivity to a measurement context may be different for each measurement context factor, each device. For example, the measurement context factor may include at least one of an external temperature, a device using time, or a user state. For example, information on a sensitivity level of each device to an external temperature, a sensitivity level of each device to a device using time, and/or a sensitivity level of each device to a user state may be pre-stored (see Table 1).

In an embodiment, the processor 210 may determine a representative electronic device from the plurality of electronic devices with which the user is in contact or which the user is wearing, based on at least part of the measurement context information, correlation information indicating a correlation with real body temperature, and sensitivity information indicating sensitivity to a measurement context.

In an embodiment, the processor 210 may select a representative electronic device based on a user state of the measurement context information.

For example, the user state may be any one of a static state and a dynamic state. The dynamic state may be a state (for example, a kinetic state) in which a change in user's body temperature is greater than or equal to a predetermined offset value, sweat is produced, or a motion is detected for a predetermined time or longer. The static state may be a state except for the dynamic state or a state in which a change in user's body temperature is maintained within a predetermined range (for example, a sitting or lying state, or a sleep state). For example, the electronic device 200 may determine whether the user state changes from the static state to the dynamic state, based on sensing information (for example, a body temperature, a motion, humidity) which is detected in at least one of the plurality of electronic devices which the user is wearing or with which the user is in contact.

The electronic device 200 may identify the user state as one of the dynamic state and the static state. When the user state is the static state, the electronic device 200 may select a representative electronic device, based on the correlation information of the plurality of electronic devices indicating a correlation with real body temperature. When the user state is the dynamic state, the electronic device 200 may select a representative electronic device, based on the sensitivity information of the plurality of electronic devices indicating sensitivity to a measurement context.

For example, the correlation with real body temperature of the earbud 203, the smart watch 202, and the smart ring 204 may be high in the order of the earbud 203 (the wearing area: ears, the correlation level: high), the smart watch 202 (the wearing area: wrist, the correlation level: medium), and the smart ring 204 (the wearing area: finger, the correlation level: low). When the user is in the static state, the electronic device 200 may select the earbud 203 (the wearing area: ears, the correlation level: high) that has the highest correlation with real body temperature as the representative electronic device.

Sensitivity to a user state may be high in the order of the earbud 203 (the wearing area: ears, the sensitivity level: high), the smart watch 202 (the wearing area: wrist, the sensitivity level: medium), and the smart ring 204 (the wearing area: finger, the sensitivity level: low). As the sensitivity to the user state is higher, the influence of a change in the user state is greater and it may be difficult to measure accurate body temperature. When the user state changes to the dynamic state in which a body temperature is dramatically changed or sweat is produced, the electronic device 200 may select the smart ring 204 (the wearing area: finger, the sensitivity level: low) that has the lowest sensitivity to the user state as the representative electronic device.

In an embodiment, the electronic device 200 may select a representative electronic device based on an external temperature of the measurement context information.

For example, when the external temperature is a first level (for example, about 15° C. to 25° C., a room temperature range that does not influence body temperature measurement), the electronic device 200 may select, as a representative electronic device, a device that has the highest correlation level from the plurality of electronic devices with which the user is in contact or which the user is wearing, with reference to the correlation with real body temperature. When the external temperature is a second level (for example, less than about 15° C., a low temperature range that influences body temperature measurement), the electronic device 200 may select, as a representative electronic device, a device that has the lowest sensitivity level to the external temperature from the plurality of electronic devices with which the user is in contact or which the user is wearing, with reference to the sensitivity to the external temperature.

In an embodiment, the electronic device 200 may select one or more valid electronic devices from the plurality of electronic devices with which the user is in contact or which the user is wearing, and then, may assess the priority of the one or more valid electronic devices by using at least one of correlation information and sensitivity information, and may select a device that has the highest priority as the representative electronic device, based on the assessment.

In an embodiment, the electronic device 200 may acquire body temperature data of respective devices (body temperature values and/or skin temperature values) from the plurality of electronic devices. The plurality of electronic devices may be being used by the user (for example, is being worn or touched).

In an embodiment, the body temperature data of respective devices may include body temperature values of respective devices (and/or skin temperature values of respective devices) which are measured by the plurality of electronic devices with which the user is in contact or which the user is wearing. The body temperature data of each device may further include device information (for example, a device ID) of the electronic device which measures a body temperature value (and/or skin temperature value).

For example, the plurality of electronic devices may include the smartphone 201 that the user is using (gripping or touching), the smart watch 202 that the user is wearing, and the earbud 203 that the user is wearing. The body temperature data of respective devices which is acquired from the plurality of electronic devices may include body temperature values (and/or skin temperature values) measured by the smart phone 201, the smart watch 202, and the earbud 203, respectively.

In an embodiment, the plurality of electronic devices which provide the body temperature data of respective devices may be the electronic device 200 (for example, the smartphone 201) and one or more external electronic devices (for example, the smart watch 202, the earbud 203). The one or more external electronic devices may transmit its own device information (for example, a device ID) and its measured body temperature value (and/or skin temperature value) to the electronic device 200 through a communication circuit (for example, the communication circuit 220 of FIG. 2) (or short-range wireless communication network connection).

In an embodiment, the plurality of electronic devices which provide the body temperature of respective devices may be two or more external electronic devices (for example, the smart watch 202, the smart band 207). The two or more external electronic devices may transmit their own device information (for example, a device ID) and their measured body temperature values (and/or skin temperature values) to the electronic device 200 through the communication circuit 220 (or short-range wireless communication connection).

In an embodiment, the electronic device 200 may identify one or more valid electronic devices among the plurality of electronic devices with which the user is in contact or which the user is wearing, based on whether the body temperature data of each device satisfies a designated condition. The electronic device 200 may select a representative electronic device from the one or more valid electronic devices based on the measurement context information. For example, the electronic device 200 may assess the priority of each of the one or more valid electronic devices, based on at least part of the measurement context information, correlation information indicating a correlation with real body temperature, and sensitivity information indicating sensitivity to a measurement context. The electronic device 200 may select a representative electronic device that has a relatively higher priority than other electronic devices from the one or more valid electronic devices, based on the assessment.

In an embodiment, the electronic device 200 may select a representative electronic device that is able to provide body temperature information relatively more accurately than the other electronic devices among the multiple electronic devices with which the user is in contact or which the user is wearing according to a real-time measurement context. The electronic device 200 may receive, through the representative electronic device, body temperature information (skin temperature value and/or body temperature value estimated from the skin temperature) that is relatively more accurate than information of the other electronic devices since the representative electronic device has a relatively higher correlation with real body temperature than the other electronic devices or is less influenced by the measurement context.

In operation 630, the electronic device 200 may acquire body temperature information of the user by using the representative electronic device determined through operation 620. The body temperature information may correspond to information on a body temperature and/or a skin temperature. For example, body temperature information acquired from the representative electronic device may correspond to information on user's temperature (for example, any one of a representative temperature, a core temperature, a reference temperature).

In an embodiment, the electronic device 200 may provide the body temperature measurement function and/or the body temperature monitoring function by using the acquired body temperature information.

For example, the electronic device 200 may receive a body temperature value (or a skin temperature value) measured by the representative electronic device, or may estimate (or calculate) a representative body temperature from the body temperature value (or skin temperature value). In another example, the electronic device 200 may accumulate and store body temperature values (or skin temperature values) measured by the representative electronic device on a predetermined time basis (for example, a daily basis). In still another example, the electronic device 200 may periodically or aperiodically (for example, when an event occurs) refine (or redetermine) the representative electronic device, and may acquire time-series continuous temperature information of the user by using one or more representative electronic devices.

In an embodiment, the electronic device 200 may provide (for example, display or output) a user interface related to the body temperature information measured through the representative electronic device.

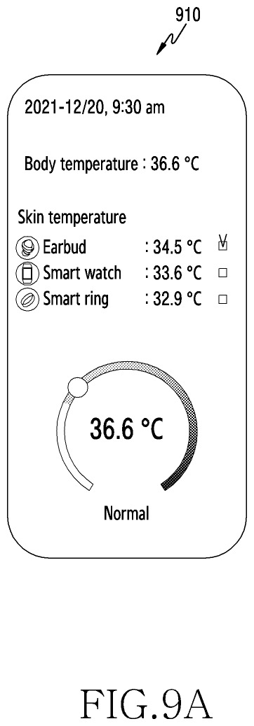

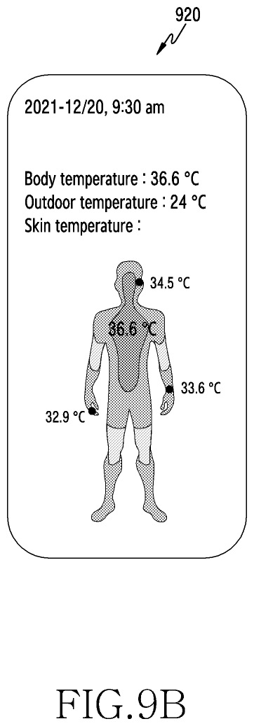

For example, the electronic device 200 may output a user interface such as a first screen 910 of FIG. 9A or a second screen 920 of FIG. 9B.

In an embodiment, the user interface related to the body temperature information may be provided in various types.

For example, the user interface related to the body temperature information may be implemented in a visual type (for example, a screen, a message, a message window), an auditory type (for example, audio, sound), a tactile type (for example, a vibration), or a hybrid type combining at least part of the aforementioned types.

For example, the electronic device 200 (for example, any one of the smartphone 201 or the smart watch 202 of FIG. 3) may output the user interface related to the body temperature information. The user interface of a visual type, an auditory type, a tactile type, or a hybrid type may be outputted to the user through the output module 250 of the electronic device 200.

In another example, the electronic device 200 (for example, any one of the smartphone 201 or the smart watch 202 of FIG. 3) may transmit information on the user interface to an external electronic device (for example, the other one of the smartphone 201 or the smart watch 202 of FIG. 3) to output the user interface (for example, a screen, a message, a voice, a vibration) through the external electronic device.

In an embodiment, the user interface related to the body temperature information may be provided in various ways.

For example, the electronic device 200 may display body temperature information obtained by tracking measurement values of the representative electronic device among body temperature data of respective devices (body temperature values and/or skin temperature values) provided through the multiple electronic devices on a daily basis through a user interface screen. The representative electronic device which provides body temperature information may be changed periodically or when the measurement context is changed or the multiple electronic devices which are targets for the user to wear (or touch) are changed.

In another example, the electronic device 200 may monitor the body temperature information through the representative electronic device, and, when an abnormal symptom is detected as a result of monitoring (for example, when the fever goes up dramatically or body temperature dramatically increases due to a disease such as a cold), the electronic device 200 may output a user interface warning of the danger of the abnormal symptom (for example, displaying a warning message window, outputting a warning sound or voice alarm, or outputting a vibration).

In another example, the electronic device 200 may accumulate user's body temperature information on a daily basis, and may provide information on a long-term trend and a characteristic parameter (for example, a health parameter such as an exercise cycle or a sleep cycle, or a medical parameter such as a woman's menstrual cycle) related to body temperature to the user interface.

In an embodiment, when there is no valid electronic device that satisfies the designated condition among the plurality of electronic devices which the user is wearing or with which the user is in contact, the electronic device 200 may provide a user interface displaying a body temperature measurement error. The user interface may include information indicating a cause of the body temperature measurement error.

In an embodiment, the electronic device 200 may determine the cause of the body temperature measurement error based on the measurement context information. The electronic device 200 may include the information indicating the cause of the body temperature measurement error in the user interface, and may output the user interface (for example, display a guidance message window or an audio guidance).

In an embodiment, the electronic device 200 may determine one or more representative electronic devices with respect to a plurality of time sections, and may acquire body temperature information measured by the one or more representative electronic devices. The electronic device 200 may provide a user interface related to the acquired body temperature information.

In an embodiment, the electronic device 200 may refine (or redetermine) the representative electronic device in response to an event for refining the representative electronic device. For example, the event may be at least one of a first event which occurs as a designated time comes, a second event which occurs as the user is in contact with or is wearing a new electronic device, and a third event which occurs as user's contact with or wearing the electronic device is released. For example, the electronic device 200 may refine (or redetermine) the representative electronic device periodically, when wearing (or contact with) of a new electronic device is detected, or when releasing of the electronic device is detected. The representative electronic device capable of measuring accurate body temperature may be changed periodically, when the measurement context is changed, or when the multiple electronic devices that the user is wearing (or touching) are changed.

FIG. 7 is another flowchart illustrating an operation method of an electronic device according to an embodiment of the disclosure.

For the convenience of explanation, it is assumed that the method of FIG. 7 is performed by the electronic device 200 of FIG. 2. However, this should not be considered as limiting. For example, the method of FIG. 7 may be performed by the processor 210 of the electronic device 200, an application (for example, a body temperature application, a health care application) executed in the electronic device 200, or the electronic device 101 or the processor 120 of FIG. 1. In a certain embodiment, at least one of operations of the method illustrated in FIG. 7 may be omitted, the sequence of some operations may be changed, or other operations may be added.

In an embodiment, the electronic device (for example, the electronic device 200 of FIG. 2) may detect a skin temperature of a measuring area, and may estimate (or calculate) a body temperature from the skin temperature. The skin temperature and/or the body temperature estimated from the skin temperature may be influenced by a measurement context. The electronic device (for example, the electronic device 200 of FIG. 2) may select a representative electronic device which is able to measure body temperature relatively more accurately than other electronic devices according to a measurement context, and may provide highly accurate body temperature information through the representative electronic device.

For example, a measurement context factor that influences body temperature measurement may include at least one of an external temperature, a device using time, a device internal temperature, a device type, a sensor arrangement structure, a measuring area or a user state.

For example, an external temperature may influence body temperature measurement. The skin is exposed to the outside and may be influenced by an external temperature. For example, as shown in FIG. 4, when the skin is exposed to a low external temperature (for example, about 10° C.), a skin temperature may decrease and a difference between the skin temperature and the core temperature may become larger than when the external temperature is a room temperature (for example, about 15-20° C.). The influence of the external temperature (sensitivity to the external temperature) may become greater toward distal body parts.

In another example, a device using time and/or a device internal temperature may influence body temperature measurement. When a body temperature is measured, the electronic device 200 may be worn on or in contact with the skin, and accordingly, a skin temperature may be influenced by the electronic device 200. The electronic device 200 and the skin may influence each other (bidirectionally), and the influence may increase as the wearing or contact time of the electronic device 200 increases.

For example, the skin temperature of a surface contacting the electronic device 200 may gradually increase due to the internal heat of the electronic device 200 (for example, heat dissipation from the processor 210 of FIG. 2 or the battery 189 of FIG. 1). As the using time of the electronic device 200 increases, a skin temperature increase rate may increase. In particular, when various applications are executed in the electronic device 200 simultaneously or a specific application is continuously used, or right after the battery of the electronic device 200 is charged, the device internal temperature of the electronic device 200 may increase significantly, and the skin temperature on the corresponding surface of the electronic device 200 may also increase to a high level. To this end, it may be difficult to measure body temperature accurately.

In another example, a device type, a sensor arrangement structure, and/or a measuring area may influence body temperature measurement. There may be various device types (for example, a draw-in type, a watch type, a ring type), sensor arrangement structures (for example, an open type, a semi-open type, a sealed type), and/or measuring areas (for example, the ears, wrist, finger) according to mechanical characteristics. A measuring area may be warmed or sealed due to a sensor arrangement structure. Heat dissipation may be difficult in a sensor contacting area (for example, the wrist, finger) or a sealed space (for example, the inside of the ear), and thus heat may be accumulated. To this end, a skin temperature may gradually increase. The degree of influence on the skin temperature (for example, sensitivity) may vary according to a size of a surface area contacting the skin or a degree of sealing in a measurement space. In addition, when sweat is produced in the warmed or sealed measuring area (for example, the wrist, finger), humidity may increase and heat dissipation may become difficult, and thus, the skin temperature may increase.

These measurement context factors may influence skin temperatures which are detected in multiple electronic devices with which the user is in contact and which the user is wearing, and/or body temperature measurement using the skin temperatures. Due to the influence of the measurement context factors, body temperatures measured in some of the multiple electronic devices may be relatively inaccurate compared to body temperatures measured in some other electronic devices.

According to an embodiment, when there are multiple electronic devices with which a user is in contact or which a user is wearing, the electronic device 200 may select a representative electronic device that is able to measure body temperature relatively more accurately than other electronic devices according to a real-time measurement context.

Referring to FIG. 7, the operation of selecting the representative electronic device may include operation 710, operation 720, and operation 730. For example, the operation of selecting the representative electronic device may correspond to operation 620 of FIG. 6.

When the user is in contact with or is wearing multiple electronic devices, there may be a need to select a device that is able to measure body temperature relatively more accurately than other electronic devices.

In an embodiment, the electronic device 200 may select a representative electronic device which is able to measure body temperately most accurately among the multiple electronic devices with which the user is in contact or which the user is wearing through operation 710 and operation 720.

Operation 710 may be identifying a valid electronic device among the multiple electronic devices with which the user is in contact or which the user is wearing.

In operation 710, the electronic device 200 (for example, the smart watch 202 of FIG. 3) may identify one or more valid electronic devices that provide valid body temperature data (body temperature value and/or skin temperature value) among the plurality of electronic devices, based on whether body temperature data of respective devices acquired from the plurality of electronic devices satisfies a designated condition.

In an embodiment, operation 710 may include operation 711, operation 713, and operation 715.

In an embodiment, when the electronic device 200 satisfies a first condition in which a body temperature value measured in a first electronic device among the plurality of electronic devices with which the user is in contact or which the user is wearing is greater than or equal to a first threshold value (for example, 30° C.), a second condition in which a device internal temperature of the first electronic device is less than or equal to a designated second threshold value (for example, 38° C.), and a third condition in which the body temperature value exceeds the device internal temperature, the electronic device 200 may identify the first electronic device as a valid electronic device. Only the device that satisfies all of the first condition (for example, a condition in which a measured body temperature value is greater than or equal to 30° C.), the second condition (for example, a condition in which a device internal temperature is less than or equal to 38° C.), and the third condition (for example, a condition in which a measured body temperature value is higher than a device internal temperature) among the plurality of electronic devices may be identified as the valid electronic device.

In operation 711, the electronic device 200 (for example, the smart watch 202 of FIG. 3) may determine whether a body temperature value measured in a specific electronic device (for example, any one of the smart watch 202, the earbud 203, the smart ring 204 of FIG. 3 that the user is wearing) is greater than or equal to 30° C. Since the human body is regulated to maintain homoeostasis, the real body temperature may not decrease to a specific temperature or lower. For example, the human's real body temperature may be maintained at about 30° C. or higher. Therefore, if the measured body temperature value is less than 30° C., the corresponding body temperature value may be an invalid value. Accordingly, if the body temperature value measured in a specific electronic device is less than about 30° C., the electronic device 200 may exclude the specific electronic device from valid electronic device classification.

When the body temperature value measured in the specific electronic device is greater than or equal to 30° C. as a result of determining in operation 711, the method may proceed to operation 713.

In operation 713, the electronic device 200 (for example, the smart watch 202) may determine whether the device internal temperature of the specific electronic device (for example, any one of the smart watch 202, the earbud 203, the smart ring 204 that the user is wearing) is less than or equal to 38° C.

The device internal temperature may increase due to a device using time and/or a structural characteristic of a measuring area (for example, a warmed or sealed structure). Typically, the device internal temperature may be maintained at 38° C. or lower. However, the device internal temperature may increase higher than 38° C. since internal heat is accumulated due to overuse of resources, battery charging or humidity. The device internal temperature higher than 38° C. may cause a temperature of the skin in contact with the device to increase. The increase of the skin temperature caused by the device internal temperature may influence a body temperature measurement result. Accordingly, when the device internal temperature of the specific electronic device is higher than 38° C., the electronic device 200 may exclude the specific electronic device from the valid electronic device classification.

When the device internal temperature of the specific electronic device is less than or equal to 38° C. as a result of determining in operation 713, the method may proceed to operation 715.

In operation 715, the electronic device 200 (for example, the smart watch 202) may determine whether the body temperature value measured in the specific electronic device (for example, any one of the smart watch 202, the earbud 203, the smart ring 204 that the user is wearing) exceeds the device internal temperature of the specific electronic device.

In general, the human body temperature may be maintained higher than the device internal temperature. A body temperature value less than or equal to the device internal temperature may not be a value corresponding to user's real body temperature but a body temperature value that is influenced by the device internal temperature through a contact surface. Accordingly, the electronic device 200 may compare the body temperature value measured in the specific electronic device with the device internal temperature of the specific electronic device, and, only when the body temperature value exceeds the device internal temperature as a result of comparing, the electronic device 200 may determine that the corresponding body temperature value is a valid value and may identify the specific electronic device as the valid electronic device. When the body temperature value measured in the specific electronic device is less than or equal to the device internal temperature, the electronic device 200 may determine the corresponding body temperature value as an invalid value, and may exclude the specific electronic device from the valid electronic device classification.

When the body temperature value measured in the specific electronic device exceeds the device internal temperature of the specific electronic device as a result of measuring in operation 715, the specific electronic device may be determined as the valid electronic device.

In an embodiment, the electronic device 200 may classify one or more electronic devices that satisfy all of the first condition, the second condition, and the third condition described above among the plurality of electronic devices with which the user is in contact or which the user is wearing as the valid electronic device.

Through operation 710, one or more valid electronic devices that provide valid body temperature values without being influenced by a measurement context such as device internal heat dissipation or a measuring area.

In an embodiment, when the user is wearing/in contact with various types of electronic devices (for example, the earbud 203, the smart watch 202, the smart ring 204), a valid electronic device may be identified among the electronic devices by using body temperature data of each device (body temperature value and/or skin temperature value).

Operation 720 may be an operation of assessing the priority of the one or more valid electronic devices which are selected through operation 710, based on the measurement context information for supporting selection of the representative electronic device.

Operation 720 may include at least one of operation 721 and operation 725.

Operation 721 may be an operation of assessing the priority based on correlation information indicating a correlation with real body temperature of the electronic devices. Operation 725 may be an operation of assessing the priority based on sensitivity information indicating sensitivity to a measurement context of the electronic devices.

In an embodiment, the electronic device 200 may perform at least one of operation 721 and operation 725 according to a measurement context (for example, at least one of an external temperature, a device using time, or a user state).

In operation 721, the electronic device 200 may assess the one or more valid electronic devices, based on the correlation with real body temperature, and may assign the priority to each valid electronic device through the assessment. For example, when the earbud 203, the smart watch 202, and the smart ring 204 are classified as valid electronic devices through operation 710, the electronic device 200 (for example, the smart watch 202) may proceed to operation 721 to determine the priority of each of the earbud 203, the smart watch 202, and the smart ring 204 with reference to a correlation level of each device.

In operation 725, the electronic device 200 may assess the one or more valid electronic devices based on sensitivity to the measurement context, and may assign the priority to each valid electronic device through the assessment. For example, when the user is wearing the earbud 203, the smart watch 202, and the smart ring 204 and the earbud 203, the smart watch 202, and the smart ring 204 are identified as valid electronic devices through operation 710, the electronic device 200 may determine the priority of each of the earbud 203, the smart watch 202, and the smart ring 204 with reference to a sensitivity level of each device to a measurement context (for example, an external temperature, a device wearing time, and/or a user state).

The correlation with real body temperature is as follows.

When a body temperature of each device is measured, a correlation between a body temperature (or skin temperature) measured at a position close to the center of the body and a real body temperature is high, and accordingly, a correlation level with real body temperature may be higher as a measuring area is closer to the center of the body. For example, the earbud 203 (the wearing area: the inside of the ear) may have a relatively higher correlation level with real body temperature than other electronic devices, and the smart watch 202 (the wearing area: wrist) may have a next high correlation level with real body temperature. The smart ring 204 (the wearing area: finger) may have a relatively low correlation level with real body temperature.

Since the correlation with real body temperature is high in the order of the ear, wrist, finger considering the measuring areas, the priority may be determined in the order of the earbud 203 (the wearing area: ear), the smart watch (the wearing area: wrist), the smart ring 204 (the wearing area: finger). A relatively high priority may be assigned to an electronic device that has low sensitivity to the wearing time among the electronic devices having the same or similar wearing areas. A relatively high priority may be assigned to an electronic device that has low sensitivity to the external temperature among the electronic devices having the same or similar measuring areas and wearing time (or use time).

Table 1 presented below is provided to explain sensitivity to measurement contexts, and shows sensitivity of various types of electronic devices (for example, the earbud 203, the smart watch 202, the smart ring 204) to measurement context factors (for example, an external temperature, a device wearing time, a user state) which influence body temperature measurement.

TABLE 1

| | Earbud | Smart watch | Smart ring |
|---|---|---|---|
| External temperature | Low | Medium | High |
| Wearing time | Medium | High | Low |
| User state | High | Medium | Low |

Referring to Table 1 presented above, the smart ring 204 among the three valid electronic devices including the earbud 203, the smart watch 202 and the smart ring 204 may be most sensitive to the external temperature. The earbud 203 is such a device type that it is worn on the ears, and may have a sensor arrangement structure of a sealed type. Since the inside of the ear is less exposed to the outside and is close to the carotid artery having a large amount of blood circulated, the earbud 203 worn inside the ear has a relatively higher correlation with real body temperature compared to the smart watch 202 and the smart ring 204, but is relatively less influenced by the external temperature (low sensitivity to the external temperature) (sensitivity level: low). Since the smart ring 204 is such a device type that it is worn on the finger and has a sensor arrangement structure of an open type, the sensitivity of the smart ring 204 to an external temperature (sensitivity level: high) may be relatively higher than the earbud 203 and the smart watch 202 due to the device characteristics of the smart ring 204.

In addition, the smart watch 202 among the three valid electronic devices including the earbud 203, the smart watch 202, and the smart ring 204 may be most sensitive to a wearing time. Since such wearable electronic devices as the earbud 203, the smart watch 202, and the smart ring 204 have a structure contacting the skin in part, the wearable electronic device and the skin may directly influence each other (bidirectionally), and the influence therebetween may increase as a wearing time increases. A skin temperature may increase due to internal heat of the wearable electronic device. The use state (for example, the type or number of running applications, an on/off state of a display, an amount of resources used) may be different for each wearable electronic device, and accordingly, the skin temperature may gradually increase through a contact surface between each wearable electronic device and the skin. For example, when the wearable electronic device executes various applications simultaneously or continuously uses a certain application, or right after a battery is charged, the device internal temperature of the wearable electronic device may gradually increase to the extent that the internal temperature influences body temperature measurement. Compared to the earbud 203 or the smart ring 204, the smart watch 202 has a relatively large area directly contacting the skin and has many components causing internal heat dissipation, and thus, may have relatively high sensitivity to a wearing time. Due to such mechanical characteristics, the sensitivity of the smart watch 202 to the wearing time (sensitivity level: high) may be relatively higher than those of the earbud 203 and the smart ring 204.

In addition, the earbud 203 among the three valid electronic devices including the earbud 203, the smart watch 202, and the smart ring 204 may be most sensitive to a user state. The sensor arrangement structure (for example, a sealed type, a semi-open type, an open type) may be different for each device type (for example, a draw-in type, a watch type, a ring type), and accordingly, a measuring area may be opened or semi-opened, or may be warmed or sealed. When the wearable electronic device has the sensor arrangement structure of the sealed type, heat dissipation in a contact area or a sealed space may become difficult and heat may be accumulated such that a skin temperature gradually increases. The degree of influence may vary according to a size of a device surface area contacting the skin or a degree of sealing of the space. In addition, when sweat is produced in the sensor arrangement structure of the sealed type, humidity in a contact area may increase and heat dissipation on the skin may become difficult, and thus, the skin temperature may increase. Due such mechanical characteristics, the degree of influence of the user state (sensitivity to the user state) may be high in the order of the earbud 203 which has the sensor arrangement structure of the sealed type, the smart watch 202 which has the sensor arrangement structure of the semi-open type, and the smart ring 204 which has the sensor arrangement structure of the open type. The sensitivity of the earbud 203 to the user state (sensitivity level: high) may be relatively higher than those of the other electronic devices.

In an embodiment, the electronic device 200 may prioritize the earbud 203, the smart watch 202 and the smart ring 204, which are valid electronic devices, based on at least one of the correlation with real body temperature or the sensitivity to the measurement context.

In an embodiment, the electronic device 200 may determine whether to perform operation 721 and operation 725 according to the measurement context (for example, an external temperature or a user state).

For example, when the external temperature is a first level (for example, a room temperature range from about 15° C. to 25° C.) or the user state is a static state, the electronic device 200 may proceed to operation 721 to assess the priority based on correlation information with real body temperature. As the correlation level with real body temperature is higher, the higher priority may be assigned. The priority may be assigned to electronic devices having the same or similar correlation levels with reference to a sensitivity level.

In another example, when the external temperature is a second level (for example, a low temperature range of about less than 15° C.) or the user state is a dynamic state, the electronic device 200 may proceed to operation 725 to assess the priority based on sensitivity information to the measurement context. As the sensitivity level is lower, the higher priority may be assigned. The priority may be assigned to electronic devices having the same or similar sensitivity levels with reference to a correlation level with real body temperature.

In an embodiment, the electronic device 200 may determine whether the user state changes from the static state to the dynamic state to assess the priority of each device. The dynamic state may correspond to a state in which there is an active change in body temperature or sweat is produced.

When the user state is the static state, a body temperature (or skin temperature) measured in a body part close to the center of the body has a high correlation with real body temperature, and accordingly, the electronic device 200 may prioritize in the order of a higher correlation with real body temperature with reference to a measuring area (for example, a wearing area). For example, the earbud 203 (the wearing area: the inside of the ear) that has a relatively higher correlation with real body temperature than other electronic devices may have the highest priority, the smart watch 202 (the wearing area: wrist) may have the intermediate priority, and the smart ring 204 (the wearing area: finger) that has a relatively lower correlation with real body temperature than other electronic devices may have the lowest priority.

When the user state changes to the dynamic state, it may be necessary to determine the priority considering the sensitivity of each electronic device 200 according to a measuring area.

For example, when there is an active change in body temperature due to fever of the user or a body activity such as exercise, user's body temperature may dramatically increase or much sweat may be produced. In this case, sensitivity according to a measuring area and a user state may be considered to assess the priority.

For example, when the measuring area (or wearing area) is the ears as in the earbud 203, a body temperature sensor may be disposed inside the ears (the sensor arrangement structure of the sealed type or closed type), and accordingly, when user's body temperature dramatically increases or much sweat is produced, heat dissipation may become difficult due to the influence thereof, and heat may be accumulated, influencing a skin temperature, and to this end, accurate body temperature may not be measured. On the other hand, when the measuring area is the finger as in the smart ring 204, body temperature measurement may be less influenced by dramatic increase in body temperature or sweat due to the sensor arrangement structure of the open type. In the case of the wrist, body temperature measurement may be moderately influenced since the device does not have a sealed structure but is in contact with the wrist.

In addition, body heat has the characteristic of spreading from the center of the human body to distal body parts. Accordingly, when there is an active change in body temperature, a skin temperature detected in the earbud 203 may be much influenced by sweat or heat in the ear, and a skin temperature in the smart ring 204 may be relatively less influenced. Since the smart watch 202 does not have a sensor arrangement structure of a sealed type but has a wide contact surface area with the skin, and may have relatively higher sensitivity to a user state than the smart ring 204. In addition, even when a fever cools down after sweating, selecting a skin temperature detected in a distal body part having low sensitivity to a user state may be a way of measuring a body temperature substantially more accurately. Accordingly, when there is an active change in body temperature, the priority may be determined in the order of the smart ring 204, the smart watch 202, and the earbud 203.

In an embodiment, the electronic device 200 may select a representative electronic device from the multiple electronic devices which the user is wearing or with which the user is in contact through two steps of operation 710 and operation 720 at operation 730.

At operation 730, the electronic device 200 may select a representative electronic device that has a relatively higher priority than the other electronic devices from the multiple electronic devices which the user is wearing or with which the user is in contact, with reference to a result of assessing the priority of each device.

For example, when the user state is the static state, the highest priority may be assigned to the earbud 203 that has the highest correlation level with real body temperature among the smart ring 204, the smart watch 202, and the earbud 203 that the user is wearing, and the earbud 203 may be selected as the representative electronic device. In another example, when there is an active change in user's body temperature (and/or sweat is produced), the highest priority may be assigned to the smart ring 204 that has the lowest sensitivity level to the user state among the smart ring 204, the smart watch 202, and the earbud 203 that the user is wearing, considering a wearing area of each device, and the smart ring 204 may be selected as the representative electronic device.

In an embodiment, the electronic device 200 may use the priority of each device according to the correlation level with real body temperature as a default priority value. For example, the earbud 203 that has the highest correlation level with real body temperature among the smart ring 204, the smart watch 202, and the earbud 203 may be assigned the highest priority as a default priority value.

In an embodiment, the electronic device 200 may determine whether it is necessary to assess the priority based on the measurement context information (for example, a user state or an external temperature).

When it is determined that it is not necessary to assess the priority, the priority of each device may be maintained by a designated default priority value according to the correlation level with real body temperature. For example, when the external temperature is the first level (for example, a room temperature range from about 15° C. to 20° C.) and the user state is the static state, it may be determined that it is not necessary to assess the priority. In this case, the earbud 203 that has the highest priority according to the default priority value among the smart ring 204, the smart watch 202, and the earbud 203 that the user is wearing may be continuously used as the representative electronic device without assessing the priority.

When it is determined that it is necessary to assess the priority, the priority may be assessed based on the measurement context information. For example, when the external temperature is the second level (for example, a low temperature range of less than about 15° C. which influences body temperature measurement), it may be determined that it is necessary to assess the priority. In this case, the highest priority may be assigned to the earbud 203 that has the lowest sensitivity level to the external temperature among the smart ring 204, the smart watch 202, and the earbud 203 that the user is wearing, such that the earbud 203 may be used as the representative electronic device. In another example, when the user state is the dynamic state or the external temperature is the third level (for example, a high-temperature range of about 30° C. or higher that influences body temperature measurement), it may be determined that it is necessary to assess the priority. In this case, the highest priority may be assigned to the smart ring 204 that has the lowest sensitivity level to the user state among the smart ring 204, the smart watch 202, and the earbud 203 that the user is wearing, such that the smart ring 204 may be used as the representative electronic device.

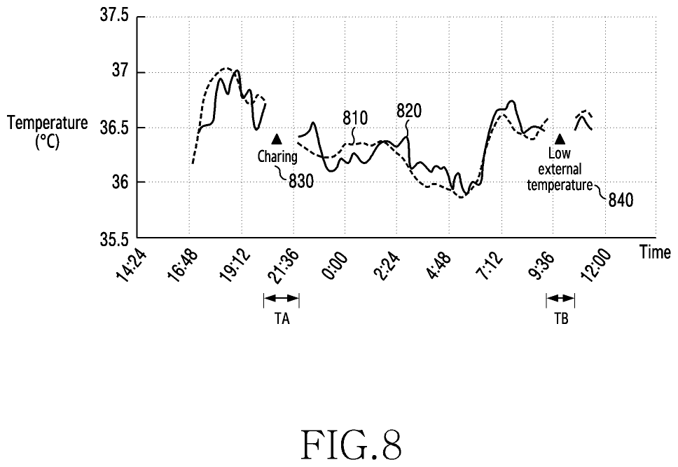

FIG. 8 is a graph illustrating body temperature information provided by an electronic device according to an embodiment of the disclosure.

In an embodiment, the electronic device 200 (for example, the smart watch 202 of FIG. 3) may receive body temperature information and may display a user interface related to the body temperature information.

FIG. 8 illustrates body temperature information according to a time axis. In the example of FIG. 8, reference numeral 820 may indicate row data measured in the smart watch 202, and reference numeral 810 may indicate body temperature information which is acquired through processing of the row data (for example, smoothing).

Each electronic device (for example, the smart watch 202) may have a regression model to measure a body temperature, and may detect a skin temperature and then may estimate (or calculate) a body temperature from the measured skin temperature by using the regression model.

A wearable electronic device such as the smart watch 202 may provide repetitive body temperature information due to its device characteristic that it is worn for a relatively long time.

In an embodiment, even when there are electronic devices which a user is wearing or with which a user is in contact, if there is no valid electronic device among the electronic devices or none of the body temperature values (or skin temperature value) measured in the electronic devices is valid, the electronic device 200 may not provide body temperature information.

For example, when the user is wearing the smart watch 202 and the smart ring 204 and is exposed to a low external temperature (for example, about 10° C.), body temperature values measured in the smart watch 202 and the smart ring 204 may be less than a designated threshold value (for example, 30° C.) due to the low external temperature. In this case, it does not mean that the real body temperature of the user falls but means that the result of measuring the body temperature is influenced by a measuring area that is greatly influenced by the external temperature (or that has high sensitivity).

When there is no valid electronic device or none of the body temperature values (or skin temperature values) measured in the electronic devices that the user is touching/wearing are valid, the electronic device 200 (for example, the smart phone 201 of FIG. 3) may determine that the result of measuring the body temperature is not valid and may not provide the corresponding body temperature information. The electronic device 200 may provide a user interface indicating a body temperature measurement error.

In an embodiment, when a body temperature measurement error occurs, the electronic device 200 may determine a cause of the body temperature measurement error, based on measurement context information.

For example, when the smart watch 202 selected as a representative electronic device switches to a charging state and it is impossible to measure a body temperature in a first section TA, the smartphone 201 providing the body temperature monitoring function may determine that a body temperature measurement error occurs. The smartphone 201 may display an indicator 830 indicating the charging state which is the cause of the body temperature measurement error without providing body temperature information during the first section TA.

The representative electronic device may be periodically refined (or redetermined). Alternatively, when the user wears a new electronic device or takes off the electronic device that the user is wearing, the representative electronic device may be changed. Alternatively, when a real-time measurement context is changed, the representative electronic device may be changed.

When the user is wearing the smart watch 202 and the smartphone 201 but valid body temperature information is not provided through the smart watch 202 and the smartphone 201 due to a low external temperature (for example, about 10° C.) in a second section TB, the smartphone 201 that provides the body temperature monitoring function may determine that a body temperature measurement error occurs. The smartphone 201 may display an indicator 840 indicating that it is impossible to measure a body temperature due to a low external temperature without providing body temperature information during the second section TB.

FIGS. 9A and 9B illustrate examples of user interfaces related to a body temperature measurement function of an electronic device according to various embodiments of the disclosure.

Referring to FIG. 9A, the electronic device 200 (for example, the smartphone 201 of FIG. 3) may display a user interface such as a first screen 910. As shown in the drawing, the first screen 910 may display a representative body temperature (for example, about 36.6° C.) and a body temperature value (or skin temperature value) of each device measured in a plurality of wearable electronic devices (for example, the earbud 203, the smart watch 202, the smart ring 204 of FIG. 3).

Referring to FIG. 9B, the electronic device 200 (for example, the smartphone 201 of FIG. 3) may display a user interface such as a second screen 920. As shown in the drawing, the second screen 920 may include information on a representative body temperature (for example, about 36.6° C.) and an external temperature (for example, about 24° C.). The second screen 920 may include an intuitive graphic user interface which shows body temperature values of respective devices (or skin temperature values) mapped onto respective body parts.

According to various embodiment, an electronic device (for example, the electronic device 200 of FIG. 2) may include memory (for example, the memory 240 of FIG. 2), a communication circuit (for example, the communication circuit 220 of FIG. 2), at least one sensor (for example, the sensor module 230 of FIG. 2), and at least one processor (for example, the processor 210 of FIG. 2) which is operatively connected with the memory, the communication circuit, and the at least one sensor. The memory may store instructions that, when executed, causes the at least one processor to: detect a plurality of electronic devices with which a user is in contact or which a user is wearing through the communication circuit or the at least one sensor; determine a representative electronic device among the plurality of electronic devices, based on measurement context information; and acquire body temperature information of the user by using the representative electronic device.

According to various embodiments, the measurement context information may include at least one of information on an external environment, information on a user state, information on a device use state, and information on a device characteristic.

According to various embodiments, correlation information indicating a correlation with a real body temperature and sensitivity information indicating sensitivity to a measurement context may be pre-stored for the plurality of electronic devices. The representative electronic device may be determined based on at least one of the correlation information and the sensitivity information.

According to various embodiments, the measurement context information may include information on a user state. When the user state is a static state, the representative electronic device may be selected based on the correlation information. When the user state is a dynamic state, the representative electronic device may be selected based on the sensitivity information.

According to various embodiments, the instructions may cause the at least one processor to: acquire body temperature data of respective devices from the plurality of electronic devices; identify one or more valid electronic devices among the plurality of electronic devices, based on whether the body temperature data of the respective devices satisfies a designated condition; and select the representative electronic device from the one or more valid electronic devices, based on the measurement context information.

According to various embodiments, when a condition in which a body temperature value measured in a first electronic device among the plurality of electronic devices is greater than or equal to a first threshold value, a condition in which a device internal temperature of the first electronic device is less than or equal to a second threshold value, and a condition in which the body temperature value exceeds the device internal temperature are satisfied, the first electronic device may be identified as the valid electronic device.

According to various embodiments, the instructions may cause the at least one processor to: assess a priority of each of the one or more valid electronic devices based on the measurement context information; and select the representative electronic device from the one or more valid electronic devices based on the assessment.

According to various embodiments, the instructions may cause the at least one processor to: when there is no valid electronic device that satisfies the designated condition, determine a cause of the body temperature measurement error based on the measurement context information; and provide a user interface including information informing the cause of the body temperature measurement error.

According to various embodiments, the instructions may cause the at least one processor to: determine one or more representative electronic devices with respect to a plurality of time sections; acquire body temperature information measured by the one or more representative electronic devices; and provide a user interface related to the body temperature information.

According to various embodiments, the instructions may cause the at least one processor to refine the representative electronic device in response to at least one of a first event which occurs as a designated time arrives, a second event which occurs as the user is in contact or wears a new electronic device, and a third event which occurs as the user's contact with or wearing the electronic device is released.

According to various embodiments, an operation method of an electronic device may include: detecting a plurality of electronic devices with which a user is in contact or which a user is wearing; determining a representative electronic device among the plurality of electronic devices, based on measurement context information; and acquiring body temperature information of the user by using the representative electronic device.

According to various embodiments, the measurement context information may include at least one of information on an external environment, information on a user state, information on a device use state, and information on a device characteristic.

According to various embodiments, correlation information indicating a correlation with a real body temperature and sensitivity information indicating sensitivity to a measurement context may be pre-stored for the plurality of electronic devices. The representative electronic device may be determined based on at least one of the correlation information and the sensitivity information.

According to various embodiments, the measurement context information may include information on a user state, and, when the user state is a static state, the representative electronic device may be selected based on the correlation information. When the user state is a dynamic state, the representative electronic device may be selected based on the sensitivity information.

According to various embodiments, determining the representative electronic device may include: acquiring body temperature data of respective devices from the plurality of electronic devices; identifying one or more valid electronic devices among the plurality of electronic devices, based on whether the body temperature data of the respective devices satisfies a designated condition; and selecting the representative electronic device from the one or more valid electronic devices, based on the measurement context information.

According to various embodiments, when a condition in which a body temperature value measured in a first electronic device among the plurality of electronic devices is greater than or equal to a first threshold value, a condition in which a device internal temperature of the first electronic device is less than or equal to a second threshold value, and a condition in which the body temperature value exceeds the device internal temperature are satisfied, the first electronic device may be identified as the valid electronic device.

According to various embodiments, selecting the representative electronic device may include: assessing a priority of each of the one or more valid electronic devices based on the measurement context information; and selecting the representative electronic device from the one or more valid electronic devices based on the assessment.

According to various embodiments, the method may further include: when there is no valid electronic device that satisfies the designated condition, determining a cause of the body temperature measurement error based on the measurement context information; and providing a user interface including information informing the cause of the body temperature measurement error.

According to various embodiments, the method may further include: determining one or more representative electronic devices with respect to a plurality of time sections; acquiring body temperature information measured by the one or more representative electronic devices; and providing a user interface related to the body temperature information.

According to various embodiments, the method may further include refining the representative electronic device in response to at least one of a first event which occurs as a designated time arrives, a second event which occurs as the user is in contact or wears a new electronic device, and a third event which occurs as the user's contact with or wearing the electronic device is released.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
memory storing one or more computer programs;
a communication circuit;
at least one sensor; and
one or more processors communicatively coupled to the memory, the communication circuit, and the at least one sensor,
wherein the one or more computer programs include computer-executable instructions that, when executed by the one or more processors individually or collectively, cause the electronic device to:
detect a plurality of electronic devices with which a user is in contact or which a user is wearing through the communication circuit or the at least one sensor,
determine a representative electronic device among the plurality of electronic devices, based on measurement context information, and
acquire body temperature information of the user by using the representative electronic device.

2. The electronic device of claim 1, wherein the measurement context information comprises at least one of information on an external environment, information on a user state, information on a device use state, and information on a device characteristic.

3. The electronic device of claim 1,
wherein correlation information indicating a correlation with a real body temperature and sensitivity information indicating sensitivity to a measurement context are pre-stored for the plurality of electronic devices, and
wherein the representative electronic device is determined based on at least one of the correlation information and the sensitivity information.

4. The electronic device of claim 3,
wherein the measurement context information comprises information on a user state,
wherein, when the user state is a static state, the representative electronic device is selected based on the correlation information, and
wherein, when the user state is a dynamic state, the representative electronic device is selected based on the sensitivity information.

5. The electronic device of claim 1, wherein the one or more computer programs further include computer-executable instructions that, when executed by the one or more processors individually or collectively, cause the electronic device to:
acquire body temperature data of respective devices from the plurality of electronic devices;
identify one or more valid electronic devices among the plurality of electronic devices, based on whether the body temperature data of the respective devices satisfies a designated condition; and
select the representative electronic device from the one or more valid electronic devices, based on the measurement context information.

6. The electronic device of claim 5, wherein, when a condition in which a body temperature value measured in a first electronic device among the plurality of electronic devices is greater than or equal to a first threshold value, a condition in which a device internal temperature of the first electronic device is less than or equal to a second threshold value, and a condition in which the body temperature value exceeds the device internal temperature are satisfied, the first electronic device is identified as the valid electronic device.

7. The electronic device of claim 5, wherein the one or more computer programs further include computer-executable instructions that, when executed by the one or more processors individually or collectively, cause the electronic device to:
assess a priority of each of the one or more valid electronic devices based on the measurement context information; and
select the representative electronic device from the one or more valid electronic devices based on the assessment.

8. The electronic device of claim 5, wherein the one or more computer programs further include computer-executable instructions that, when executed by the one or more processors individually or collectively, cause the electronic device to:
when there is no valid electronic device that satisfies the designated condition, determine a cause of a body temperature measurement error based on the measurement context information; and
provide a user interface including information informing the cause of the body temperature measurement error.

9. The electronic device of claim 1, wherein the one or more computer programs further include computer-executable instructions that, when executed by the one or more processors individually or collectively, cause the electronic device to:
determine one or more representative electronic devices with respect to a plurality of time sections;
acquire body temperature information measured by the one or more representative electronic devices; and
provide a user interface related to the body temperature information.

10. The electronic device of claim 1, wherein the one or more computer programs further include computer-executable instructions that, when executed by the one or more processors individually or collectively, cause the electronic device to refine the representative electronic device in response to at least one of a first event which occurs as a designated time arrives, a second event which occurs as the user is in contact or wears a new electronic device, and a third event which occurs as the user's contact with or wearing the electronic device is released.

11. An operation method of an electronic device, the operation method comprising:
detecting a plurality of electronic devices with which a user is in contact or which a user is wearing;
determining a representative electronic device among the plurality of electronic devices, based on measurement context information; and
acquiring body temperature information of the user by using the representative electronic device.

12. The operation method of claim 11, wherein the measurement context information comprises at least one of information on an external environment, information on a user state, information on a device use state, and information on a device characteristic.

13. The operation method of claim 11,
wherein correlation information indicating a correlation with a real body temperature and sensitivity information indicating sensitivity to a measurement context are pre-stored for the plurality of electronic devices, and
wherein the representative electronic device is determined based on at least one of the correlation information and the sensitivity information.

14. The operation method of claim 13,
wherein the measurement context information comprises information on a user state,
wherein, when the user state is a static state, the representative electronic device is selected based on the correlation information, and
wherein, when the user state is a dynamic state, the representative electronic device is selected based on the sensitivity information.

15. The operation method of claim 11, wherein determining the representative electronic device comprises:
acquiring body temperature data of respective devices from the plurality of electronic devices;
identifying one or more valid electronic devices among the plurality of electronic devices, based on whether the body temperature data of the respective devices satisfies a designated condition; and
selecting the representative electronic device from the one or more valid electronic devices, based on the measurement context information.

16. The operation method of claim 15, wherein, when a condition in which a body temperature value measured in a first electronic device among the plurality of electronic devices is greater than or equal to a first threshold value, a condition in which a device internal temperature of the first electronic device is less than or equal to a second threshold value, and a condition in which the body temperature value exceeds the device internal temperature are satisfied, the first electronic device is identified as the valid electronic device.

17. The operation method of claim 15, further comprising:
assessing a priority of each of the one or more valid electronic devices based on the measurement context information; and
selecting the representative electronic device from the one or more valid electronic devices based on the assessment.

18. The operation method of claim 15, further comprising:
when there is no valid electronic device that satisfies the designated condition, determine a cause of a body temperature measurement error based on the measurement context information; and
provide a user interface including information informing the cause of the body temperature measurement error.

19. One or more non-transitory computer-readable storage media storing one or more computer programs including computer-executable instructions that, when executed by one or more processors of an electronic device individually or collectively, cause the electronic device to perform operations, the operations comprising:
detecting a plurality of electronic devices with which a user is in contact or which a user is wearing;
determining a representative electronic device among the plurality of electronic devices, based on measurement context information; and
acquiring body temperature information of the user by using the representative electronic device.

20. The one or more non-transitory computer-readable storage media of claim 19, wherein the measurement context information comprises at least one of information on an external environment, information on a user state, information on a device use state, and information on a device characteristic.
